(19) United States
(12) Patent Application Publication
(10) Pub. No.: US 2025/0065091 A1
(43) Pub. Date: Feb. 27, 2025

(54) INGESTIBLE DEVICES, ASSEMBLIES, AND METHODS FOR DELIVERING A THERAPEUTIC PREPARATION INTO A GASTROINTESTINAL TRACT

(71) Applicant: Rani Therapeutics, LLC, San Jose, CA (US)

(72) Inventor: Mir Imran, Los Altos Hills, CA (US)

(73) Assignee: Rani Therapeutics, LLC, San Jose, CA (US)

(21) Appl. No.: 18/825,266

(22) Filed: Sep. 5, 2024

Related U.S. Application Data
(63) Continuation of application No. PCT/US2023/014670, filed on Mar. 7, 2023.
(60) Provisional application No. 63/317,798, filed on Mar. 8, 2022.

(51) Int. Cl. A61M 31/00

(52) U.S. Cl. CPC A61M 31/002; A61M 2202/0007

(57) ABSTRACT

An ingestible device for delivering a therapeutic preparation into a lumen wall or a surrounding tissue of a gastrointestinal (GI) tract of a subject includes an enclosure, a support, an arm, a delivery assembly, an extender, an actuator, and a release. The support is disposed in the enclosure and defines a longitudinal axis of the device. The delivery assembly includes a delivery structure coupled to the arm, and a therapeutic preparation disposed in the delivery structure. The extender is coupled to at least one of the support or the delivery assembly. The extender is structured to extend along the longitudinal axis to substantially align the delivery structure relative to the lumen wall. The actuator is coupled to the delivery assembly. The release is coupled to the actuator and is activated in response to a condition in the GI tract to cause actuation of the actuator. When the release is activated, the actuator causes the arm to pivot relative to the support such that the delivery assembly extends outwardly away from the longitudinal axis toward the lumen wall to deliver the therapeutic preparation from the delivery structure into the lumen wall or surrounding tissue.

### CROSS-REFERENCE TO RELATED APPLICATIONS

This application is a continuation of International Patent Application No. PCT/US2023/014670, filed Mar. 7, 2023, which claims the benefit of and priority to U.S. Provisional Application No. 63/317,798, filed on Mar. 8, 2022, the entire contents of which are hereby incorporated by reference herein.

### BACKGROUND

Generally speaking, a therapeutic agent such as a drug may be administered to a subject by ingestion or through parenteral injection (e.g., subcutaneously, intramuscularly, or intravenously) to provide a desired therapeutic effect. However, these routes of administration have some disadvantages. For example, some therapeutic agents such as large (macro) molecules are not suitable for delivery by ingestion because of enzymatic breakdown of these molecules in the gastrointestinal (GI) tract of a subject. Other types of therapeutic agents may otherwise be poorly tolerated within the GI tract resulting in low systemic uptake. With parenteral injections, subjects may experience pain and inconvenience with administration which can significantly impact compliance and quality of life.

Thus, there is a need for improved devices, assemblies, and methods for delivering a therapeutic agent into the GI tract which address one or more of the above disadvantages.

### SUMMARY

Embodiments of the present disclosure relate generally to ingestible devices, assemblies, and methods for effectively delivering one or more therapeutic agents into a GI lumen wall or surrounding tissue thereof (e.g., a peritoneum or a peritoneal cavity) of a subject.

In one aspect, an ingestible device for delivering a therapeutic preparation into a lumen wall or a surrounding tissue of a GI tract of a subject includes an enclosure, a support, an arm, a delivery assembly, an extender, an actuator, and a release. The support is disposed in the enclosure and defines a longitudinal axis of the device. The delivery assembly includes a delivery structure coupled to the arm, and a therapeutic preparation disposed in the delivery structure. The extender is coupled to at least one of the support or the delivery assembly. The extender is structured to extend along the longitudinal axis so as to substantially align the delivery structure relative to the lumen wall. The actuator is coupled to the delivery assembly. The release is coupled to the actuator and is activated in response to a condition in the GI tract so as to cause actuation of the actuator. In one or more embodiments, when the release is activated, the actuator causes the arm to pivot relative to the support such that the delivery assembly extends outwardly away from the longitudinal axis toward the lumen wall to deliver the therapeutic preparation from the delivery structure into the lumen wall.

In another aspect, a delivery assembly for delivering a therapeutic preparation into a lumen wall or a surrounding tissue of a GI tract of a subject includes a delivery structure and a therapeutic preparation. The delivery structure is structured to be coupled to an arm and contains the therapeutic preparation therein. The delivery assembly is structured to extend outwardly away from the support such that the delivery structure is positioned adjacent, or in contact with, a surface of the lumen wall. The delivery assembly is structured to eject the therapeutic preparation from the delivery structure into the lumen wall.

In another aspect, a method of delivering a therapeutic preparation into a lumen wall or a surrounding tissue of a GI tract of a subject includes: administering to a subject an ingestible device, where the ingestible device includes an enclosure, a support, an arm, a delivery assembly, an extender, an actuator, and a release. The support defines a longitudinal axis of the device. The arm is pivotably coupled to the support. The delivery assembly includes a delivery structure coupled to the arm, and a therapeutic preparation disposed in the delivery structure. The extender is coupled to at least one of the support or the delivery assembly. The actuator is coupled to the delivery assembly. The release is coupled to the actuator. The actuator is actuated in response to activation of the release. The extender extends along the longitudinal axis to align the delivery structure relative to the lumen wall upon actuation of the actuator. The arm pivots relative to the support such that the delivery assembly extends outwardly away from the longitudinal axis toward the lumen wall. The therapeutic preparation is ejected from the delivery structure into the lumen wall or surrounding tissue thereof.

In one or more embodiments of the foregoing aspects, the actuator is structured to generate an axial force along the longitudinal axis to cause the arm to pivot.

In one or more embodiments of the foregoing aspects, the actuator is structured to generate a rotational force about the longitudinal axis to cause the arm to pivot.

In one or more embodiments of the foregoing aspects, the support includes a plurality of sections movably coupled together to define a telescoping arrangement such that when the release is activated, the plurality of sections move relative to each other such that the support extends axially along the longitudinal axis.

In one or more embodiments of the foregoing aspects, the actuator is coupled to the delivery assembly and the extender such that when the release is activated, the actuator substantially simultaneously causes the delivery assembly to extend outwardly away from the longitudinal axis and the extender to extend along the longitudinal axis.

In one or more embodiments of the foregoing aspects, the enclosure defines an interior, where the support, the arm, the delivery assembly, the extender, the actuator, and the release are disposed within the interior.

In one or more embodiments of the foregoing aspects, the enclosure is structured as a capsule including a first segment and a second segment, where the first segment is detachably coupled to the second segment. At least one of the actuator, the support, or the extender is structured to push the first and second segments apart.

In one or more embodiments of the foregoing aspects, the release is a first release and the actuator is a first actuator. The delivery structure further includes a second release operably coupled to a second actuator for actively delivering the therapeutic preparation. The second release is structured to cause the second actuator to eject the therapeutic preparation from the delivery structure into the GI lumen wall or surrounding tissue after the first actuator causes the delivery assembly to extend outwardly away from the support.

In one or more embodiments of the foregoing aspects, the second release is selectively exposed to fluid in the GI lumen in response to movement of the delivery assembly so as to activate the second release.

In one or more embodiments of the foregoing aspects, the second release is selectively moved in response to movement of the delivery assembly so as to activate the second release.

In one or more embodiments of the foregoing aspects, the first release has a first degradation rate and the second release has a second degradation rate, where the second degradation rate is less than the first degradation rate.

In one or more embodiments of the foregoing aspects, the delivery structure includes a holder coupled to the arm and a container coupled to the holder, where the container is structured to contain the therapeutic preparation therein.

In one or more embodiments of the foregoing aspects, the container includes a body, a first seal, and a second seal. The body includes a first open end, a second open end opposite the first open end, and a side wall defining an interior for containing the therapeutic preparation. The first seal is coupled at the first open end and the second seal is coupled at the second open end such that the interior is substantially sealed.

In one or more embodiments of the foregoing aspects, the holder includes a protrusion disposed within an interior portion of the holder. The container is slidably coupled to, and at least partially disposed in, the interior portion of the holder adjacent the protrusion. The delivery structure is structured such that when the container contacts the lumen wall in response to the delivery assembly extending outwardly away from the longitudinal axis, the container moves inward toward the protrusion to cause the protrusion to eject the therapeutic preparation from the container into the lumen wall.

In one or more embodiments of the foregoing aspects, the holder defines a piston chamber and the delivery structure includes a piston slidably disposed in the piston chamber.

In one or more embodiments of the foregoing aspects, the holder further defines a reaction chamber, and the delivery structure further includes an igniter, a charge, and an electrical circuit. The igniter and the charge are disposed in the reaction chamber. The electrical circuit is operatively coupled to the igniter. The igniter ignites the charge in response to a signal received from the electrical circuit so as to generate a force within the reaction chamber to cause the piston to eject the therapeutic preparation from the container into the lumen wall.

In one or more embodiments of the foregoing aspects, the holder further defines a reaction chamber, and the delivery structure further includes a first reactant disposed in the reaction chamber, a second reactant disposed in the piston chamber below the piston, and a release mechanism coupled to the holder. Upon activation of the release mechanism, the reaction chamber is fluidly coupled with the piston chamber to mix the first reactant with the second reactant to cause a chemical reaction so as to generate a force to cause the piston to eject the therapeutic preparation from the container into the lumen wall.

In one or more embodiments of the foregoing aspects, the delivery structure further includes a spring coupled to the piston and a release mechanism coupled to the spring. Upon activation of the release mechanism, the spring expands to cause the piston to eject the therapeutic preparation from the container into the lumen wall.

In one or more embodiments of the foregoing aspects, the delivery structure includes a retaining feature for temporarily retaining the delivery structure relative to the lumen wall.

In one or more embodiments of the foregoing aspects, the device further includes a plurality of arms pivotably coupled together in a criss-cross manner to define a scissor mechanism, where the arm is one of the plurality of arms. One or more of the plurality of arms may be slidably coupled to the support.

In one or more embodiments of the foregoing aspects, the actuator includes a spring. The spring is held in a compressed state by the release such that upon activation of the release, the spring expands along the longitudinal axis to cause the arm to pivot relative to the support.

In one or more embodiments of the foregoing aspects, the actuator includes an inflatable member. The inflatable member also functions as the extender. The inflatable member includes a plurality of reactants disposed therein, where the reactants are temporarily separated from each other by the release. The release is coupled to the inflatable member such that upon activation of the release, the plurality of reactants mix together to cause a chemical reaction within the inflatable member to create a gas to cause the inflatable member to expand and thereby cause the arm to pivot relative to the support.

In one or more embodiments of the foregoing aspects, the actuator includes a housing defining a piston chamber, and the support includes a piston slidably disposed within the piston chamber.

In one or more embodiments of the foregoing aspects, the housing further defines a reaction chamber, and the actuator further includes an igniter and a charge disposed in the reaction chamber, and an electrical circuit operatively coupled to the igniter. Upon activation of the release, the electrical circuit generates a signal to cause the igniter to ignite the charge so as to generate a force within the reaction chamber to cause the piston and the support to move along the longitudinal axis relative to the housing to thereby cause the arm to pivot relative to the support.

In one or more embodiments of the foregoing aspects, the actuator further includes a hydrogel disposed in the piston chamber. The release is coupled to the housing. The housing includes a plurality of openings to provide a fluid path between the chamber and the GI lumen environment. Upon activation of the release, fluid from the GI lumen environment enters the chamber through one or more of the plurality of openings to cause the hydrogel to expand within the chamber to move the piston and the support to thereby cause the arm to pivot relative to the support.

In one or more embodiments of the foregoing aspects, the actuator includes a motor. The motor is a rotary motor and the support includes a threaded portion rotatably coupled to the rotary motor. The release is disposed over electrical contacts of the motor such that upon activation of the release, an electrical short is created across the electrical contacts to cause the motor to rotate the support relative to the motor and the support to translate along the longitudinal axis to thereby cause the arm to pivot relative to the support.

In one or more embodiments of the foregoing aspects, the device further comprises a plurality of delivery assemblies pivotably coupled to the support.

The foregoing general description and following detailed description are provided by way of example and are intended to provide further explanation of the disclosure as claimed, without limiting the disclosure or the claims. Other objects, advantages, and novel features will be readily apparent to those skilled in the art from the following brief description of the drawings and detailed description of the disclosure.

### BRIEF DESCRIPTION OF THE DRAWINGS

FIG. 1 illustrates an embodiment of an ingestible device in block diagrammatic form.

FIG. 2A illustrates a cross-section of an embodiment of an ingestible device in capsule form. The device is shown in a first state prior to the device reaching a desired location in the GI tract for delivering a therapeutic preparation.

FIG. 2B illustrates the device of FIG. 2A in a second state after the device reaches the desired location in the GI tract.

FIG. 3A illustrates a cross-section of an embodiment of an ingestible device including an actuator in the form of a spring. The device is shown in a first state prior to the device reaching a desired location in the GI tract for delivering a therapeutic preparation.

FIG. 3B illustrates the device of FIG. 3A in a second state after the device reaches the desired location in the GI tract.

FIG. 4A illustrates a cross-section of an embodiment of an ingestible device including an actuator in the form of an inflatable member. The device is shown in a first state prior to the device reaching a desired location in the GI tract for delivering a therapeutic preparation.

FIG. 4B illustrates the device of FIG. 4A in a second state after the device reaches the desired location in the GI tract.

FIG. 5A illustrates a cross-section of an embodiment of an ingestible device including an actuator in the form of a piston, an igniter, and a charge. The device is shown in a first state prior to the device reaching a desired location in the GI tract for delivering a therapeutic preparation.

FIG. 5B illustrates a cross-section of the device of FIG. 5A in a second state after the device reaches the desired location in the GI tract.

FIG. 6A illustrates a cross-section of an embodiment of an ingestible device including an actuator in the form of a rotary motor. The device is shown in a first state prior to the device reaching a desired location in the GI tract for delivering a therapeutic preparation.

FIG. 6B illustrates the device of FIG. 6A in a second state after the device reaches the desired location in the GI tract.

FIG. 7A illustrates a cross-section of an embodiment of an ingestible device including an actuator in the form of a piston and a hydrogel. The device is shown in a first state prior to the device reaching a desired location in the GI tract for delivering a therapeutic preparation.

FIG. 7B illustrates the device of FIG. 7A in a second state after the device reaches the desired location in the GI tract.

FIG. 8A illustrates a cross-section of an embodiment of an ingestible device including a pivotable arm. The device is shown in a first state prior to the device reaching a desired location in the GI tract for delivering a therapeutic preparation.

FIG. 8B illustrates the device of FIG. 8A in a second state after the device reaches the desired location in the GI tract.

FIG. 9A illustrates a cross-section of an embodiment of an ingestible device including a pivotable arm and a movable wedge. The device is shown in a first state prior to the device reaching a desired location in the GI tract for delivering a therapeutic preparation.

FIG. 9B illustrates the device of FIG. 9A in a second state after the device reaches the desired location in the GI tract.

FIG. 10A illustrates a cross-section of an embodiment of a delivery structure including a protrusion for delivering a therapeutic preparation into a lumen wall of a GI tract. The delivery structure is shown in a first state prior to delivering the therapeutic preparation.

FIG. 10B illustrates the delivery structure of FIG. 10A in a second state after the protrusion ejects the therapeutic preparation from the delivery structure into the lumen wall.

FIG. 11A illustrates a cross-section of an embodiment of a delivery structure including a piston, an igniter, and a charge for actively delivering a therapeutic preparation into a lumen wall of a GI tract. The delivery structure is shown in a first state prior to delivering the therapeutic preparation.

FIG. 11B illustrates the delivery assembly of FIG. 11A in a second state after the piston ejects the therapeutic preparation from the delivery structure into the lumen wall.

FIG. 12A illustrates a cross-section of an embodiment of a delivery structure including a spring and a piston for actively delivering a therapeutic preparation into a lumen wall of a GI tract. The delivery structure is shown in a first state prior to delivering the therapeutic preparation.

FIG. 12B illustrates the delivery structure of FIG. 12A in a second state after the piston ejects the therapeutic preparation from the delivery structure into the lumen wall.

FIG. 13A illustrates a cross-section of an embodiment of a delivery structure including a plurality of reactants and a piston for actively delivering a therapeutic preparation into a lumen wall of a GI tract. The delivery structure is shown in a first state prior to delivering the therapeutic preparation.

FIG. 13B illustrates the delivery structure of FIG. 13A in a second state after the piston ejects the therapeutic preparation from the delivery structure into the lumen wall.

FIG. 14A illustrates a front cross-sectional view of an ingestible device including a plurality of delivery assemblies. The device is shown in a first state prior to the device reaching a desired location in the GI tract for delivering a therapeutic preparation.

FIG. 14B illustrates the device of FIG. 14A in a second state after the device reaches the desired location.

FIG. 15 illustrates a method of delivering a therapeutic preparation into a lumen wall or surrounding tissue of a GI tract of a subject by oral administration of an ingestible device.

### DETAILED DESCRIPTION

Before discussing details of the ingestible devices, assemblies, and methods of the present disclosure, a few conventions are provided for the convenience of the reader.

When used in the present disclosure, the terms “e.g.,” “such as”, “for example”, “for an example”, “for another example”, “examples of”, “by way of example”, and “etc.” indicate that a list of one or more non-limiting example(s) precedes or follows; it is to be understood that other examples not listed are also within the scope of the present disclosure.

As used herein, the singular terms “a,” “an,” and “the” may include plural references unless the context clearly dictates otherwise. Reference to an object in the singular is not intended to mean “one and only one” unless explicitly so stated, but rather “one or more.”

As used herein, a phrase in the form “A/B” or in the form “A and/or B” means (A), (B), or (A and B); a phrase in the form “at least one of A, B, or C” means (A), (B), (C), (A and B), (A and C), (B and C), or (A, B, and C).

The term “in an embodiment” or a variation thereof (e.g., “in another embodiment” or “in one embodiment”) refers herein to use in one or more embodiments, and in no case limits the scope of the present disclosure to only the embodiment as illustrated and/or described. Accordingly, a component illustrated and/or described herein with respect to an embodiment can be omitted or can be used in another embodiment (e.g., in another embodiment illustrated and described herein, or in another embodiment within the scope of the present disclosure and not illustrated and/or not described herein).

The term “component” refers herein to one item of a set of one or more items that together make up a device, a composition, or a system under discussion. A component may be in a solid, powder, gel, plasma, fluid, gas, or other constitution. For example, a device may include multiple solid components which are assembled together to structure the device and may further include a fluid component that is disposed in the device. For another example, a composition may include a single component, or two or more components which are mixed together to make the composition. A composition may be in the form of a fluid, a slurry, a powder, or a solid (e.g., in a condensed or a consolidated form such as a tablet or microtablet). A device or system can include one or more compositions and/or one or more other components.

The term “design” or a grammatical variation thereof (e.g., “designing” or “designed”) refers herein to characteristics intentionally incorporated based on, for example, estimates of tolerances (e.g., component tolerances and/or manufacturing tolerances) and estimates of environmental conditions expected to be encountered (e.g., temperature, humidity, external or internal ambient pressure, external or internal mechanical pressure, stress from external or internal mechanical pressure, age of product, or shelf life, or, if introduced into a body, physiology, body chemistry, biological composition of fluids or tissue, chemical composition of fluids or tissue, pH, species, diet, health, gender, age, ancestry, disease, or tissue damage); it is to be understood that actual tolerances and environmental conditions before and/or after delivery can affect characteristics so that different components, devices, compositions, or systems with a same design can have different actual values with respect to those characteristics. Design encompasses also variations or modifications before or after manufacture.

The term “structured” or a grammatical variation thereof (e.g., “structure” or “structuring”) refers herein to a component, device, composition, or system that is manufactured according to a concept or design or variations thereof or modifications thereto (whether such variations or modifications occur before, during, or after manufacture) whether or not such concept or design is captured in a writing.

The term “body” refers herein to an mammalian body, unless the context clearly dictates otherwise.

The term “subject” refers herein to a body into which an embodiment of the present disclosure is, or is intended to be, delivered. For example, with respect to humans, a subject may be a patient under treatment of a health care professional. The terms “individual,” “subject,” and “patient” may be used interchangeably herein, and refer to any individual animalia subject (e.g., bovine, canine, feline, equine, or human). In specific embodiments, the subject, individual, or patient is a human.

The term “fluid” refers herein to a liquid or gas, and encompasses moisture and humidity. The term “fluidic environment” refers herein to an environment in which one or more fluids are present.

The term “ingest” or a grammatical variation thereof (e.g., “ingesting”, “ingestion,” or “ingested”) refers herein to taking into the stomach, whether by swallowing or by other means of depositing into the stomach (e.g., by depositing into the stomach by endoscope or depositing into the stomach via a port).

The term “degrade” or a grammatical variation thereof (e.g., “degrading”, “degraded”, “degradable”, and “degradation”) refers herein to weakening, partially degrading, or fully degrading, such as by dissolution, chemical degradation (including biodegradation), decomposition, chemical modification, mechanical degradation, or disintegration, which encompasses also, without limitation, dissolving, crumbling, deforming, shriveling, or shrinking. The term “non-degradable” refers to an expectation that degradation will be minimal, or within a certain acceptable design percentage, for at least an expected duration in an expected environment.

The term “degradation rate” or a grammatical variation thereof (e.g., “rate of degradation”) refers herein to a rate at which a material degrades. A designed degradation rate of a material in a particular implementation can be defined by a rate at which the material is expected to degrade under expected conditions (e.g., in physiological conditions) at a target delivery site. A designed degradation time for a particular implementation can refer to a designed time to complete degradation or a designed time to a partial degradation sufficient to accomplish a design purpose (e.g., breach). Accordingly, for example, a designed degradation time can be specific to a component and/or specific to expected conditions at a target delivery site. A designed degradation time can be short or long and can be defined in terms of approximate times, maximum times, or minimum times.

The term “substantially” is used herein to describe and account for small variations which may result from, for example, a manufacturing or assembly process. For example, when used in conjunction with a numerical value, the terms can refer to a variation in the value of less than or equal to +10%.

The term “lumen” refers herein to the inside space of a tubular structure. Examples of lumens in a body include arteries, veins, and tubular cavities within organs.

The term “lumen wall” refers to a wall of a lumen, where the wall includes all layers from an inner perimeter to an outer perimeter of the lumen, such as, with respect to lumens in a body, the mucosa, submucosa, muscularis, serosa, and an outer wall of the lumen, with the constituent blood vessels and tissues.

The term “gastrointestinal tract” or “GI tract” refers herein to the intake/expulsion system of a body including, for example, the mouth, pharynx, esophagus, stomach, pylorus, small intestine, cecum, large intestine, colon, rectum, anus, and valves or sphincters therebetween.

The term “GI lumen” refers generally to any lumen of the GI tract (e.g., a lumen of the esophagus, stomach, small intestine, large intestine, or colon) and the term “GI lumen wall” refers to a lumen wall of a GI lumen.

As used herein, the terms “comprising”, “comprise”, “comprises”, “includes”, and “including” are intended to mean that the compositions and methods include the recited elements, but do not exclude others.

As used herein, the phrase “therapeutically effective amount” with reference to a medication means a dose of the medication that provides the specific pharmacological effect for which the drug is administered in a subject in need of such treatment. It is emphasized that a therapeutically effective amount of a medication will not always be effective in treating a target condition in every individual subject, even though such dose is deemed to be a therapeutically effective amount by those of skill in the art. Those skilled in the art can adjust what is deemed to be a therapeutically effective amount in accordance with standard practices as needed to treat a specific subject. A therapeutically effective amount may vary based on, for example, the age and weight of the subject, and/or the subject's overall health, and/or the severity of the condition of the subject being treated.

Referring generally to the Figures, disclosed herein are embodiments relating to ingestible devices, assemblies, and methods for delivering a therapeutic preparation into a lumen wall or surrounding tissue thereof (e.g., a peritoneum or a peritoneal cavity) of a GI tract of a subject. The ingestible devices are advantageously structured to temporarily protect the therapeutic preparation from degradation within at least a portion of the GI tract until delivery. The delivery into the lumen wall or surrounding tissue allows for systemic uptake of one or more therapeutic agents of the therapeutic preparation. Various types of therapeutic agents are discussed below; examples of such agents are macromolecules that are normally unsuitable for delivery by ingestion. In this manner, the disclosed ingestible devices, assemblies, and methods address many of the disadvantages associated with conventional oral and parenteral routes of administration.

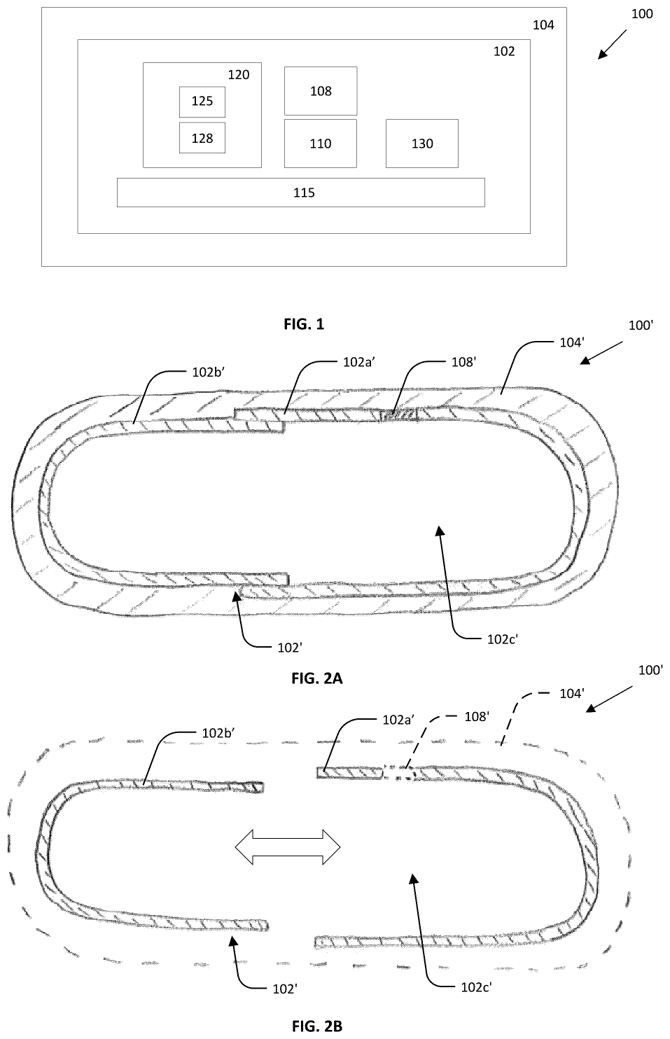

FIG. 1 illustrates in block diagrammatic form an example of an ingestible device 100 according to one or more embodiments of the present disclosure. Ingestible device 100 includes an enclosure 102, an optional outer coating 104, a release 108, an actuator 110, a support 115, a delivery assembly 120, a therapeutic preparation 125, a delivery structure 128, and an extender 130. In one or more embodiments, support 115 may form part of at least one of delivery assembly 120, actuator 110, or extender 130.

According to a non-limiting example, enclosure 102 (or optionally enclosure 102 and/or outer coating 104) can degrade at a desired location in the GI tract of a subject for delivery of therapeutic preparation 125, as described in more detail below. As described in more detail below, in response to at least partial degradation of enclosure 102 and/or outer coating 104, release 108 can be activated to actuate actuator 110. Actuator 110 can then cause at least one of support 115 or extender 130 to extend axially so as to align delivery structure 128 relative to the GI lumen wall of a subject. Actuator 110 may either substantially simultaneously, or later after actuating extender 130, cause delivery assembly 120 to move outwardly toward the GI lumen wall to deliver therapeutic preparation 125 from delivery structure 128 into the lumen wall or surrounding tissue where one or more therapeutic agents of therapeutic preparation 125 can release into the subject's bloodstream for systemic uptake.

Enclosure 102 and/or outer coating 104 are structured to allow for ingestion of ingestible device 100, and to temporarily protect the contents of ingestible device 100 from degradation within one or more portions of the GI tract of a subject. Enclosure 102 may take a variety of different forms and shapes, such as a swallowable capsule (e.g., a size 00 capsule, a size 000 capsule, or other size capsule), or any other structure that is suitable for ingestion by a subject and can house or contain one or more of the components of ingestible device 100. In one or more embodiments, enclosure 102 includes two or more segments coupled (e.g., press-fit) together to define enclosure 102. For example, enclosure 102 may be structured as a capsule including a first segment at least partially overlapping a second segment in a press-fit arrangement to define enclosure 102. The first and second segments may be detachably coupled together so as to allow for separation of the two segments by, for example, the application of an internal force (e.g., from extension of support 115, delivery assembly 120, and/or extender 130.) Release 108, actuator 110, support 115, delivery assembly 120, therapeutic preparation 125, delivery structure 128, and extender 130 are each structured to be contained within an interior of enclosure 102. Release 108 may optionally be located outside of the interior of enclosure 102, such as on an outer portion of enclosure 102. In other embodiments, outer coating 104 may itself function as a container or a protective layer for containing/protecting from degradation one or more of release 108, actuator 110, support 115, delivery assembly 120, therapeutic preparation 125, delivery structure 128, and extender 130 therein without enclosure 102.

In one or more embodiments, enclosure 102 (or optionally enclosure 102 and/or outer coating 104) can degrade under certain conditions. Further, different portions of enclosure 102 may be structured to degrade under different conditions or at different degradation rates depending on a target site within the GI tract for delivering therapeutic preparation 125. For example, a portion of, or all of, enclosure 102 may be constructed of a material that degrades in water (e.g., in the presence of water in the form of humidity or moisture in an ambient environment, such as within the body) and/or degrades when exposed to solutions with a pH level above a particular threshold or within a particular range (e.g., a pH level associated with a desired location or portion of the GI tract.) In other embodiments, enclosure 102 degrades in response to a temperature threshold. In other embodiments, enclosure 102 is substantially non-degradable or includes substantially non-degradable portions.

Outer coating 104 optionally covers a portion of, or all of, enclosure 102. In other embodiments, outer coating 104 directly covers one or more of release 108, actuator 110, support 115, delivery assembly 120, therapeutic preparation 125, delivery structure 128, and extender 130 without enclosure 102. In these embodiments, outer coating 104 can function as a protective layer for temporarily protecting one or more components of ingestible device 100 from degradation within the GI tract without a separate enclosure 102. Outer coating 104 may include a single layer or multiple layers. The various layers may be formed of the same material or a combination of different materials. An example of outer coating 104 is an enteric coating, such as an enteric coating that degrades in water at a given rate and/or degrades when exposed to solutions with a pH level above a particular threshold or within a particular range. Another example of outer coating 104 is a protective coating (e.g., wax), such as a coating which protects a portion of an outer surface of enclosure 102 from coming into contact with fluids or tissues (e.g., bodily tissue or fluids.)

In one or more embodiments, degradation of enclosure 102 and/or outer coating 104 allows fluid (e.g., bodily fluid in the stomach or in the intestine) to enter into an interior of enclosure 102/outer coating 104 to activate release 108. Alternatively, release 108 is located on an outer portion of enclosure 102 and degradation of outer coating 104 may expose release 108 on a surface of enclosure 102 to facilitate activation of release 108. Alternatively, release 108 is located on a portion of enclosure 102 without outer coating 104, and release 108 may be structured to degrade at a different rate and/or under different conditions in the GI tract than enclosure 102. Enclosure 102 and/or outer coating 104 may define one or more degradation areas for localized degradation of enclosure 102 and/or outer coating 104 so as to, for example, allow for controlled degradation and separation of enclosure 102 such that at least one of support 115, delivery assembly 120, or extender 130 can push enclosure 102 apart by application of a force within enclosure 102. For example, outer coating 104 may be selectively applied only to certain areas of enclosure 102 (e.g., on the ends of enclosure 102) to expose a selected portion of enclosure 102 (e.g., a middle portion of enclosure 102 between the ends), thereby defining an area of enclosure 102 that can degrade at a faster rate and/or degrade sooner than other areas of enclosure 102. This controlled degradation of enclosure 102 may allow for more consistent separation of enclosure 102 to thereby allow for delivery of therapeutic preparation 125 into the GI lumen wall from delivery structure 128 (e.g., by creating a substantially unobstructed area between separated portions of enclosure 102 for deployment of delivery structure 128.)

Release 108 is a chemical, mechanical, electrical, electro-mechanical, electro-chemical, chemo-mechanical, or electro-mechanical-chemical structure. In one or more embodiments, release 108 is structured to be activated (e.g., degrade, release, move, open) in response to a condition in the GI tract. For example, release 108 may be structured to degrade in water, such that release 108 may degrade upon contact with fluid in the GI tract. For another example, release 108 may be structured to degrade at or above a particular pH level or within a range of pH levels associated with a location in the GI tract (e.g., a pH in the stomach, a pH in the intestine.) In these and other embodiments, release 108 may be made from a biodegradable material, such as an enteric material. Additionally or alternatively, release 108 may be structured as a latch, a clip, a cover, a plug, a coating, or any other structure that moves, opens, or otherwise releases in response to a condition in the GI tract (e.g., a pH.) Release 108 may be formed from a single material or a combination of materials. Release 108 may include one or more components. In embodiments in which a plurality of components are included in release 108, the components may be co-located (e.g., co-axially) or may be physically separated from each other.

Release 108 is coupled to actuator 110 such that upon activation of release 108, actuator 110 can cause extender 130 to extend axially and/or can cause delivery assembly 120 to extend outwardly for delivering therapeutic preparation 125 from delivery structure 128 into the GI lumen wall. In one or more embodiments, release 108 is located within an interior of enclosure 102/outer coating 104. In other embodiments, release 108 is disposed on a portion (e.g., outer surface) of enclosure 102.

Ingestible device 100 may include a plurality of releases (e.g., release 108 and/or other release mechanisms) that activate under different conditions and/or activate at different rates to cause sequential actuation of various components of ingestible device 100. In one or more embodiments, ingestible device 100 may be structured to selectively expose and/or cause activation of one or more additional release mechanisms in response to movement of a component of ingestible device 100 after activation of release 108. For example, ingestible device 100 may include a first release (e.g., release 108) associated with actuator 110 and a second release (e.g., a release mechanism) associated with a component of delivery assembly 120, such as an actuating mechanism of delivery structure 128. In this example, first release may be structured to activate sooner and/or at a faster rate than second release, such that activation of first release can cause actuator 110 to move one or more components of delivery assembly 120 into a desired position relative to a GI lumen wall prior to activation of second release. Additionally or alternatively, movement of delivery assembly 120 (e.g., pivotable movement of one or more arms) may expose or move second release so as to activate second release. Activation of second release can then cause delivery structure 128 to actively deliver therapeutic preparation 125 into the GI lumen wall when delivery assembly 120 is positioned proximate to the GI lumen wall.

According to another example, first release may be associated with a first actuator and second release may be associated with a second actuator. First actuator may be coupled to extender 130 and second actuator may be coupled to delivery assembly 120 such that activation of first release can cause extender 130 to extend and align delivery structure 128 relative to a GI lumen wall prior to activation of second release. Activation of second release can then cause second actuator to move one or more components of delivery assembly 120 relative to the GI lumen wall for delivering therapeutic preparation 125 from delivery structure 128 into the GI lumen wall.

Actuator 110 is a chemical, mechanical, electrical, electro-mechanical, electro-chemical, chemo-mechanical, or electro-mechanical-chemical structure. In one or more embodiments, actuator 110 is structured to actuate at least one of extender 130, delivery assembly 120, or support 115 in response to activation of release 108. Actuator 110 may be structured to cause extender 130 to extend axially to align delivery structure 128 relative to a GI lumen wall. Additionally or alternatively, actuator 110 may be structured to cause one or more components of delivery assembly 120 to pivot, so as to cause delivery structure 128 to move proximate to a GI lumen wall to deliver therapeutic preparation 125 from delivery structure 128 into the lumen wall. Additionally or alternatively, actuator 110 may be structured to cause support 115 to extend, adjust, and/or move axially along a longitudinal axis defined by support 115. Actuator 110 may be structured to cause substantially simultaneous actuation of any combination of support 115, delivery assembly 120, or extender 130. Actuator 110 may include one or more components. In embodiments in which a plurality of components are included in actuator 110, the components may be co-located or may be physically separated from each other. In one or more embodiments, ingestible device 100 may include a plurality of actuators (e.g., including actuator 110) or actuating mechanisms. For example, ingestible device 100 may include separate actuators associated with any one of, or a combination of, extender 130, delivery assembly 120, and delivery structure 128.

Support 115 is a mechanical structure for holding and providing structural support to at least one of actuator 110, delivery assembly 120, therapeutic preparation 125, delivery structure 128, or extender 130 within enclosure 102. In one or more embodiments, support 115 may form part of at least one of actuator 110, delivery assembly 120, or extender 130. Support 115 is a generally elongated member and may include one or more sections that define a longitudinal axis of ingestible device 100. In embodiments including two or more sections, the sections may be movably (e.g., slidably) coupled together to define an extendible (e.g., telescoping) arrangement in which support 115 can extend axially, for example, in response to actuation of actuator 110. Support 115 may be movable relative to other components of ingestible device 100 to, for example, cause actuation of delivery assembly 120 and/or extender 130. Support 115 may include one or more components. In embodiments in which a plurality of components are included in support 115, the components may be co-located (e.g., co-axially) or may be physically separated from each other.

Delivery assembly 120 is a chemical, mechanical, electrical, electro-mechanical, electro-chemical, chemo-mechanical, or electro-mechanical-chemical structure. In one or more embodiments, delivery assembly 120 is coupled to support 115 via one or more arms pivotably coupled to support 115. The arms may form part of device 100 and/or of delivery assembly 120. Delivery assembly 120 further includes delivery structure 128 for containing therapeutic preparation 125 and for delivering therapeutic preparation 125 into the GI lumen wall. One or more components of delivery assembly 120 are structured to pivot relative to support 115 in response to actuation of actuator 110 to cause delivery assembly 120 to extend outwardly away from support 115 via one or more arms toward a GI lumen wall for delivery of therapeutic preparation 125. For example, in one or more embodiments, device 100 includes a plurality of arms pivotably coupled together in a criss-cross manner to define a scissor mechanism. In other embodiments, device 100 includes a single pivotable arm pivotably coupled to support 115. The pivotable arm is structured to pivot outwardly away from support 115 about a single pivot axis on support 115. One or more components of delivery assembly 120 may be movably coupled to support 115 to allow translatory movement of delivery assembly 120 relative to support 115. Ingestible device 100 may include one or more delivery assemblies 120. Delivery assembly 120 may include one or more components. In embodiments in which a plurality of components are included in delivery assembly 120, the components may be co-located or may be physically separated from each other.

Therapeutic preparation 125 is a preparation including one or more components where the preparation is intended for a therapeutic, diagnostic, or other biological purpose. Therapeutic preparation 125 may be in a liquid form, a powder form, or a condensed or a consolidated form such as a tablet or microtablet. A component of therapeutic preparation 125 can be, for example, a therapeutic agent, such as a pharmacologically active agent (e.g., a drug, a protein, a peptide, a polypeptide, an antibody, an oligonucleotide), a DNA or SiRNA transcript, a cell, a cytotoxic agent, a vaccine or other prophylactic agent, a nutraceutical agent, a vasodilator, or a vasoconstrictor, a delivery enhancing agent, a delay agent, an excipient, a diagnostic agent, or a substance for cosmetic enhancement. Therapeutic preparation 125 may include a therapeutically effective amount of the therapeutic agent, or suitable amounts of other components to achieve a desired therapeutic effect in a subject. Ingestible device 100 may include one or more therapeutic preparations 125 disposed in each delivery assembly 120 for delivery into the GI lumen wall. In embodiments including more than one therapeutic preparation 125, each therapeutic preparation 125 may include the same components, or may include different components (e.g., a different pharmacologically active agent) so as to provide a particular therapeutic effect.

In one or more embodiments, therapeutic preparation 125 includes a structure for inserting therapeutic preparation 125 into the GI lumen wall. For example, therapeutic preparation 125 may include a biodegradable structure defining a cavity for containing one or more components of therapeutic preparation 125 therein. The biodegradable structure may further include a tapered end (e.g., a spike, a spear, or otherwise pointed end) for piercing and inserting therapeutic preparation 125 into the GI lumen wall or surrounding tissue thereof. Additionally or alternatively, therapeutic preparation 125 may be deposited (e.g., dipped, sprayed) as a coating onto a surface of the biodegradable structure. The biodegradable structure can degrade within the tissue of the GI lumen wall, or tissue surrounding the GI lumen wall, to release the therapeutic agent of therapeutic preparation 125 from the biodegradable structure into the subject's blood stream. For example, the biodegradable structure may comprise a biodegradable polymer (e.g., polyethylene glycol (PEG)), cellulose, and/or a sugar such as maltose. In other embodiments, therapeutic preparation 125 may itself be formed into a shaped structure having a tapered end for piercing the GI lumen wall without a separate biodegradable structure. In these embodiments, therapeutic preparation 125 is a rigid or semi-rigid structure having a defined shape and sufficient rigidity to pierce and be inserted into the GI lumen wall or surrounding tissue thereof where one or more therapeutic agents of therapeutic preparation 125 can release into the subject's bloodstream. It should be appreciated that ingestible device 100 may include any one of, or a combination of, the various forms of therapeutic preparation 125 discussed above.

Therapeutic preparation 125 is contained within a sealed container to substantially preserve therapeutic preparation 125 for delivery into the GI lumen wall to achieve a desired therapeutic effect in a subject. In one or more embodiments, the sealed container may comprise a body defining an interior for containing therapeutic preparation 125 therein and one or more degradable seals (e.g., foil, film) coupled to the body for substantially sealing the interior so as to temporarily protect therapeutic preparation 125 from degrading within the GI lumen before delivery into the lumen wall. The sealed container forms part of delivery structure 128 of delivery assembly 120. Therapeutic preparation 125 is structured to pierce and penetrate through a degradable seal of the sealed container for delivery from delivery structure 128 into the GI lumen wall.

Delivery structure 128 is a chemical, mechanical, electrical, electro-mechanical, electro-chemical, chemo-mechanical, or electro-mechanical-chemical structure. Delivery structure 128 forms part of delivery assembly 120. Delivery structure 128 is structured to hold the sealed container containing therapeutic preparation 125. In one or more embodiments, delivery structure 128 may be structured to deliver therapeutic preparation 125 from the sealed container into the GI lumen wall. For example, delivery structure 128 may include a holder having a protrusion. The sealed container may be movably (e.g., slidably) coupled to the holder adjacent the protrusion such that when delivery assembly 120 is actuated, the sealed container contacts a GI lumen wall with sufficient force such that the sealed container is pushed back against the protrusion by the lumen wall to pierce a degradable seal of the container and cause the protrusion to eject therapeutic preparation 125 from the container into the GI lumen wall. In other words, the delivery structure 128 delivers therapeutic preparation 125 without a separate release/actuating mechanism because the delivery structure 128 relies on the return force from contacting the GI lumen wall.

Additionally or alternatively, delivery structure 128 may be structured to deliver therapeutic preparation 125 from the sealed container into the GI lumen wall by using a separate actuating mechanism. For example, delivery structure 128 may include a separate actuating mechanism (e.g., a spring, a piston, an igniter/charge, a gas) and a corresponding release mechanism for triggering the actuating mechanism to actively apply a force to a surface of therapeutic preparation 125 such that therapeutic preparation 125 is ejected from the container into the GI lumen wall. The release mechanism may be structured to activate sequentially with release 108. For example, the release mechanism may be structured to be activated in response to movement of delivery assembly 120 (e.g., by selectively exposing the release mechanism to GI fluid.) Additionally or alternatively, the release mechanism may have a different degradation rate than release 108 to cause activation of the release mechanism after delivery assembly 120 has been actuated. In one or more embodiments, delivery structure 128 may further include a retaining feature (e.g., hook, barb) for temporarily holding delivery structure 128 relative to the GI lumen wall to facilitate delivery of therapeutic preparation 125 into the lumen wall.

It should be appreciated that ingestible device 100 may include any one of, or a combination of, various delivery structures 128 for delivering therapeutic preparation 125 into a GI lumen wall.

Extender 130 is a chemical, mechanical, electrical, electro-mechanical, electro-chemical, chemo-mechanical, or electro-mechanical-chemical structure. Extender 130 is an adjustable structure. In one or more embodiments, extender 130 is coupled to support 115 and is structured to extend (e.g., expand, elongate, inflate) at least axially along the longitudinal axis defined by support 115, so as to help align delivery assembly 120 relative to a GI lumen wall. For example, extender 130 may extend axially along a longitudinal axis defined by support 115 such that the longitudinal axis is substantially aligned with a longitudinal axis of a GI lumen of a subject to thereby allow for delivery structure 128 to be oriented substantially orthogonal to the GI lumen wall upon actuation of actuator 110. The substantially orthogonal alignment of delivery structure 128 relative to the GI lumen wall may help to facilitate sufficient penetration of therapeutic preparation 125 into the lumen wall tissue. Extender 130 may extend in response to actuation of actuator 110 or in response to a separate actuator. Extender 130 may include one or more components. In embodiments in which a plurality of components are included in extender 130, the components may be co-located or may be physically separated from each other.

One or more components of ingestible device 100 (e.g., capsule 102, release 108, actuator 110, support 115, delivery assembly 120, delivery structure 128, extender 130) may be formed from, or otherwise include one or more biodegradable materials to facilitate degradation of such components to, for example, allow for passage through the remainder of the intestinal tract of a subject after delivery of therapeutic preparation 125. Examples of biodegradable materials that may be suitable for use with various components of ingestible device 100 include, for example, hydroxypropyl methylcellulose (HPMC), polyvinyl acetate (PVA), lactide, glycolide, lactic acid, glycolic acid, par-dioxanone, trimethylene carbonate, caprolactone, and mixtures and copolymers thereof.

The preceding description is an overview of ingestible device 100. The following description with reference to FIGS. 2A-15 relates to examples of various features and aspects of ingestible device 100 for delivering therapeutic preparation 125 into a GI lumen wall. These examples are illustrative of, and not limiting on, ingestible device 100.

Referring to FIG. 2A, a side cross-sectional view of an ingestible device 100′ in capsule form is illustrated in a first state prior to ingestible device 100′ reaching a desired location in a GI tract for delivering therapeutic preparation 125. Ingestible device 100′ is shown without any components disposed therein, but it should be appreciated that ingestible device 100′ may include at least one of release 108, actuator 110, support 115, delivery assembly 120, therapeutic preparation 125, delivery structure 128, or extender 130 disposed therein. As shown in FIG. 2A, ingestible device 100′ includes an enclosure 102′ (an embodiment of enclosure 102) structured as a swallowable capsule having a generally cylindrical body and hemispherical ends defined by a first segment 102a′ and a second segment 102b′. First segment 102a′ is detachably coupled to second segment 102b′ in a partially overlapping arrangement with first segment 102a′ partially overlapping second segment 102b′. First segment 102a′ may be press-fit onto second segment 102b′. First segment 102a′ and second segment 102b′ may be sealed together along an outer seam between the two segments 102a′ and 102b′. First segment 102a′ and second segment 102b′ cooperatively define an interior 102c′ for containing one or more of release member 108, actuator 110, delivery assembly 120, therapeutic preparation 125, delivery structure 128, and extender 130 therein. Enclosure 102′ includes an optional release 108′ (an embodiment of release member 108) extending through a portion of first segment 102a′ which may be structured to cause actuation of an actuator (e.g., actuator 110) disposed in enclosure 102′. Ingestible device 100′ further includes outer coating 104′ disposed over an exterior surface of enclosure 102′ to temporarily protect release 108′ and enclosure 102′ from degradation through one or more portions of the GI tract.

Referring to FIG. 2B, ingestible device 100′ is shown in a second state when ingestible device 100′ has reached the desired location in the GI tract to trigger degradation of outer coating 104′ and/or of enclosure 102′. For example, one or more portions of outer coating 104′ and/or one or more portions of enclosure 102′ may be structured to degrade at a threshold pH value, or within a range of pH values, associated with a desired location in the stomach or the intestine (e.g., the small intestine) to deliver therapeutic preparation 125 into a lumen wall of a subject. Release 108′ may be structured to degrade under the same or different conditions as outer coating 104′ and/or enclosure 102′ so as to trigger alignment of delivery structure 128 for delivery of therapeutic preparation 125. For example, degradation of release 108′ may cause actuation of actuator 110 to thereby cause at least one of extender 130 or delivery assembly 120 to extend. In response, at least one of support 115, delivery assembly 120, or extender 130 may cause, or assist with, axial separation of first segment 102a′ from second segment 102b′ as represented generally by a block arrow in FIG. 2B. Separation of first segment 102a′ from second segment 102b′ can, advantageously, help to facilitate alignment of delivery structure 128 (as discussed above) and actuation of delivery assembly 120 (e.g., by creating a substantially unobstructed area for deployment of delivery structure 128) so as to help facilitate delivery of therapeutic preparation 125 into a GI lumen wall.

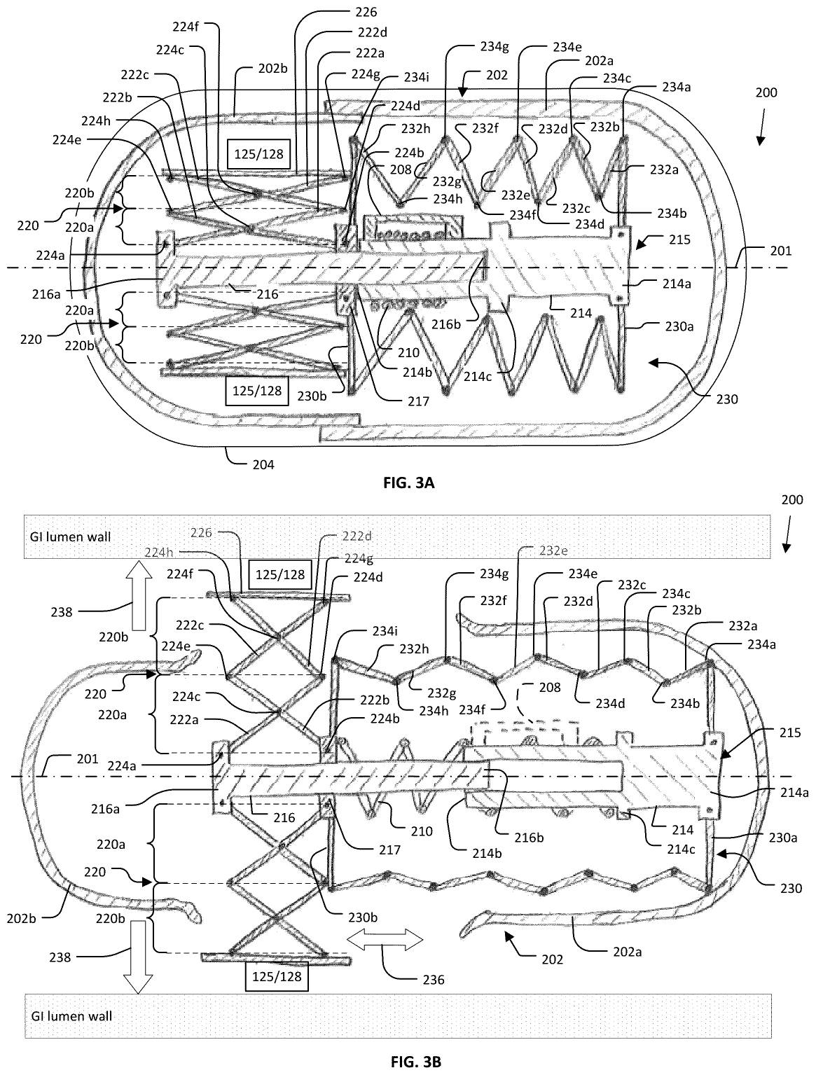

Referring now to FIG. 3A, a side cross-sectional view of an ingestible device 200 including an actuator in the form of a spring 210 (an embodiment of actuator 110) is shown. Ingestible device 200 is illustrated in a first state prior to ingestible device 200 reaching a desired location in a GI tract for delivery of therapeutic preparation 125 into the GI lumen wall. Ingestible device 200 includes an enclosure 202 (an embodiment of enclosure 102) and an outer coating 204 (an embodiment of outer coating 104.) Enclosure 202 is structured as a capsule having a first segment 202a detachably coupled to a second segment 202b.

Ingestible device 200 further includes a support 215 (an embodiment of support 115) disposed within an interior of enclosure 202. Support 215 defines a generally longitudinal axis 201 of ingestible device 200. Support 215 includes a first support section 214 and a second support section 216. First support section 214 is movably coupled to second support section 216 so as to define a generally telescoping arrangement. In the example shown, first support section 214 is partially disposed over, and slidably coupled to, second support section 216. First support section 214 and second support section 216 have a complementary shape, or include portions that are complementary to each other to permit relative translatory (e.g., sliding) movement. First support section 214 defines a first support section first end 214a and a first support section second end 214b. Likewise, second support section 216 defines a second support section first end 216a and a second support section second end 216b. First support section 214 further includes a protrusion 214c disposed on an outer surface thereof. Protrusion 214c defines an engagement surface for engaging spring 210 upon expansion of spring 210, the details of which are discussed in the paragraphs that follow.

Still referring to FIG. 3A, spring 210 is disposed over an outer portion of first support section 214. Spring 210 is shown as a coil spring, but it should be appreciated that spring 210 may be any type of spring (e.g., helical), or any other type of biasing member that can function in a similar manner as a spring. In the position shown in FIG. 3A, spring 210 is held in a compressed state by a release 208 (an embodiment of release 108) coupled to spring 210. For example, release 208 may surround at least a portion of, or all of, spring 210 to hold one or more coils of spring 210 in a compressed state. Ingestible device 200 further includes a slidable member 217 slidably coupled to second support section 216. Slidable member 217 may be a hub, a sleeve, a piston, a protrusion, a gear, or any other feature that can be movably coupled to second support section 216. For example, second support section 216 may include a slot, a channel, an outer surface, or other complementary feature to movably receive slidable member 217. In the first state shown in FIG. 3A, slidable member 217 is positioned adjacent first support section second end 214b. At least a portion of second support section 216 is structured to permit sliding movement of slidable member 217 relative to second support section 216. Slidable member 217 and second support section 216 may comprise one or more materials having a sufficiently low coefficient of friction to permit relative sliding movement.

At least one of first support section 214 or second support section 216 may include a stop feature to limit the amount of relative axial travel between the two sections and prevent them from completely separating from each other.

Ingestible device 200 further includes a delivery assembly 220 (an embodiment of delivery assembly 120) pivotably coupled to support 215. In the embodiment shown in FIG. 3A, ingestible device 200 includes two delivery assemblies 220 positioned on opposite sides of support 215, although it should be appreciated that ingestible device 200 may include one or more, two or more, three or more, or four or more delivery assemblies 220 according to other embodiments. Device 200 includes a plurality of arms 222a-222d pivotably coupled together in a criss-cross manner to define a plurality of criss-cross sections 220a, 220b of a scissor mechanism (similar to a scissor lift) which, as discussed below, is structured to extend outwardly away from longitudinal axis 201 toward a lumen wall in the GI tract of a subject to deliver therapeutic preparation 125 into the lumen wall. Arms 222a-222d are shown as substantially linear members, but arms 222a-222d may be substantially non-linear, or include substantially non-linear portions, according to other embodiments.

As shown in FIG. 3A, device 200 includes two criss-cross sections 220a, 220b pivotably coupled together. First criss-cross section 220a includes a first arm 222a pivotably coupled to second support section 216 at, or near, second support section first end 216a by a first pivot joint 224a. First criss-cross section 220a further includes a second arm 222b pivotably coupled to slidable member 217 by a second pivot joint 224b. First arm 222a and second arm 222b are positioned in a criss-cross manner relative to each other and are pivotably coupled together by a third pivot joint 224c at a location between the ends of each arm 222a, 222b to define first criss-cross section 220a. Second criss-cross section 220b is pivotably coupled to first criss-cross section 220a at, or near, distal ends of first criss-cross section 220a. For example, second criss-cross section 220b includes a third arm 222c pivotably coupled to an end of first arm 222a by a fourth pivot joint 224d. Second criss-cross section 220b further includes a fourth arm 222d pivotably coupled to an end of second arm 222b by a fifth pivot joint 224e. Third arm 222c and fourth arm 222d are positioned in a criss-cross manner relative to each other and are pivotably coupled together by a sixth pivot joint 224f at a location between the ends of each arm 222c, 222d to define second criss-cross section 220b. The pivot joints 224a-224f may each be structured as a pin, a ball and socket joint, a hinge, or any other (passive or active) joint that can permit relative pivotable movement between the respective arms 222a-222d. As noted above, device 200 may include any number of criss-cross sections (e.g., criss-cross sections 220a, 220b and additional or fewer criss-cross sections which are similar to or alternative to criss-cross sections 220a, 220b).

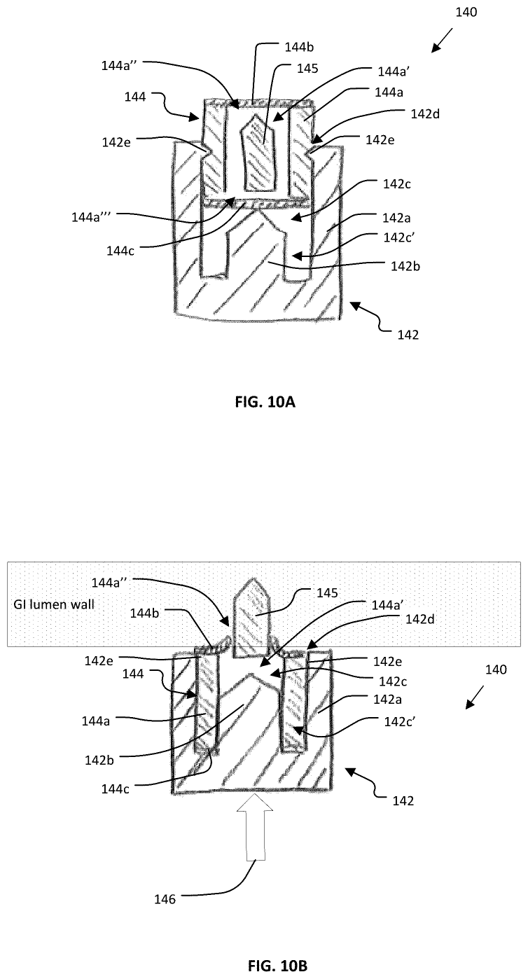

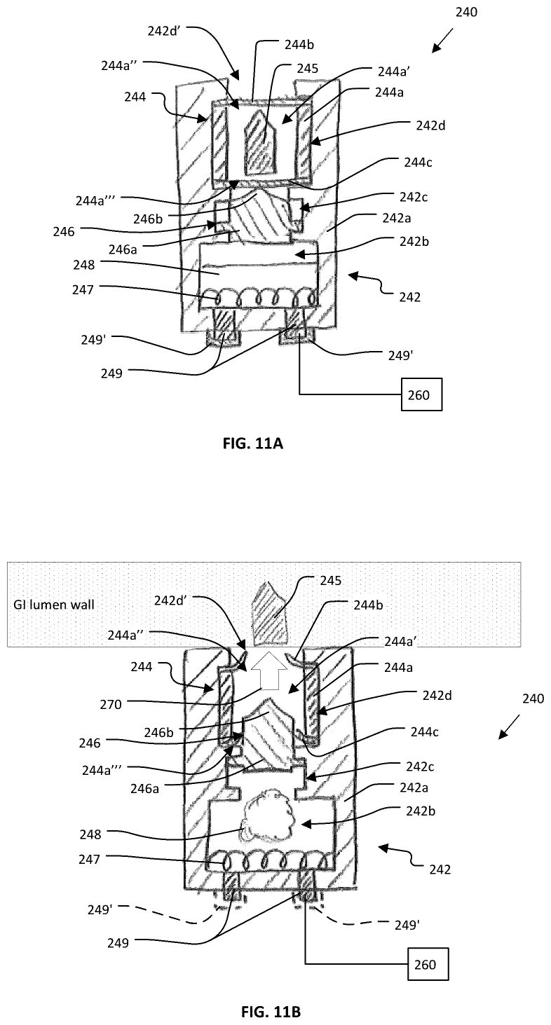

Delivery assembly 220 further includes a platform 226 pivotably and slidably coupled to second criss-cross section 220b at, or near, distal ends of second criss-cross section 220b by seventh and eight pivot joints 224g, 224h respectively. For example, pivot joints 224g, 224h may each be structured as a pin, a ball and socket joint, a hinge, or other joint, and platform 226 may include one or more rails, channels, slots, or other complementary features for engaging with pivot joints 224g, 224h to permit slidable and pivotable movement of second criss-cross section 220b relative to platform 226. Platform 226 is generally planar and is structured to provide support to a delivery structure 128, which is coupled to or integrally formed with platform 226. Delivery structure 128 and therapeutic preparation 125 are shown in block diagrammatic form in FIGS. 3A-3B, but delivery structure 128 is structured to contain one or more therapeutic preparations 125 therein and to eject therapeutic preparation 125 from delivery structure 128 into a lumen wall or surrounding tissue thereof. The details of various embodiments of delivery structure 128 are discussed below with reference to FIGS. 10A-13B.

As discussed below with reference to FIG. 3B, the arms 222a-222d are structured to pivot relative to each other and relative to platform 226 such that each delivery assembly 220 adjusts from a collapsed state (shown in FIG. 3A) to an extended state (shown in FIG. 3B) so as to facilitate delivery of therapeutic preparation 125 from delivery structure 128 into a GI lumen wall. Each delivery assembly 220 may be structured to extend outwardly until delivery structures 128 contact the GI lumen wall. Additionally or alternatively, delivery assemblies 220 may be structured to extend outward from support 215 a particular distance, or within a range of distances by, for example, selection of the size (e.g., length) of arms 222a-222d, selection of the number of criss-cross sections to include in each delivery assembly 220, and/or selection of spring 210 (e.g., spring stiffness, amount of spring travel.)

Still referring to FIG. 3A, ingestible device 200 further includes an extender 230 (an embodiment of extender 130) coupled to support 215 and to slidable member 217. Extender 230 is structured to extend axially along longitudinal axis 201, so as to help orient ingestible device 200 within the GI lumen of a subject to facilitate delivery of therapeutic preparation 125 in an approximately perpendicular manner relative to the GI lumen wall. For example, extender 230 includes a plurality of frame members 232a-232h pivotably coupled together in a zig-zag pattern by respective pivot joints 234a-234i to define an expandable wire frame or skeleton-like structure. Pivot joints 234a-234i may be structured similarly as pivot joints 224a-224f discussed above. Extender 230 may include any number of frame members. Extender 230 may define a generally elongated structure having any suitable cross-sectional shape (e.g., circular, triangular, pentagonal, hexagonal.) Additionally or alternatively, extender 230 may be a hollow, unitary structure comprising one or more concertinaed sides that can expand (similar to a bellows or an accordion.) Extender 230 defines an extender first end 230a and an extender second end 230b. Extender first end 230a is coupled to first support section 214 at, or near, first support section first end 214a. Extender second end 230b is coupled to slidable member 217 (e.g., via second pivot joint 224b.) Extender 230 is shown disposed over release 208 and actuator 210, however, it should be appreciated that release 208 may be accessible through one or more openings of extender 230 to permit activation of release 208 (e.g., degradation resulting from contact with fluid in the GI tract.)

Referring now to FIG. 3B, ingestible device 200 is shown in a second state when ingestible device 200 has reached a desired location in the GI tract of a subject to deliver therapeutic preparation 125 from delivery structures 128 into the GI lumen wall of the subject. As shown in FIG. 3B, enclosure 202 and/or outer coating 204 are partially (and may be fully) degraded as a result of ingestible device 200 having reached the desired location in the GI tract, which, for example, is associated with a selected or threshold pH value to trigger degradation of enclosure 202 and/or outer coating 204. As a result, fluid in the GI tract is permitted to reach an interior of enclosure 202 to contact release 208 so as to activate (e.g., degrade) release 208. In response to activation of release 208, spring 210 is released from the compressed state (shown in FIG. 3A) to an expanded state (shown in FIG. 3B.) Expansion of spring 210 along longitudinal axis 201 causes first support section 214 and second support section 216 to move axially away from each other, thereby extending an axial length of support 215. Further, expansion of spring 210 causes slidable member 217 to translate (e.g., slide) axially along second support section 216 toward second support section first end 216a. For example, upon expansion of spring 210, one end of spring 210 contacts protrusion 214c on first support section 214 to push first support section 214 axially away from second support section 216. A second, opposite end of spring 210 contacts slidable member 217 to push slidable member 217 along second support section 216 toward second support section first end 216a.

Axial movement of slidable member 217 toward second support section first end 216a and axial movement of first support section 214 away from second support section 216 also causes extender 230 to extend axially along longitudinal axis 201. For example, extender first end 230a is at a fixed axial position relative to first support section 214. Extender second end 230b is coupled to slidable member 217. Thus, the translatory movement of slidable member 217 toward second support section first end 216a and the axial movement of first support section 214 away from second support section 216 causes extender 230 to extend axially in two directions along longitudinal axis 201. As shown in FIG. 3B, the axial movement of first support section 214 and/or extender 230 can, advantageously, help to push apart first and second segments 202a, 202b of capsule 202 to thereby assist in creating a substantially unobstructed area for deployment of delivery structures 128 toward the GI lumen wall. Moreover, extension of extender 230 and extension of support 215 helps to substantially align longitudinal axis 201 with a longitudinal axis of the GI lumen at the particular location in the lumen at which extender 230 and support 215 are actuated. Substantially aligning longitudinal axis 201 with the longitudinal axis of the GI lumen advantageously may help to orient delivery structures 128 to be substantially orthogonal relative to the GI lumen wall so as to help to facilitate delivery of therapeutic preparation 125 into the GI lumen wall.

Axial movement of slidable member 217 along longitudinal axis 201 further causes the arms 222a-222d to pivot relative to each other such that each delivery assembly 220 extends outwardly away from longitudinal axis 201 toward the GI lumen wall. For example, as shown in FIG. 3B, the translatory movement of second arm 222b toward first arm 222a via slidable member 217 causes second arm 222b to pivot about second pivot joint 224b and first arm 222a to pivot about first pivot joint 224a, since first arm 222a is at a fixed axial position relative to support 215. This movement causes third arm 222c and fourth arm 222d to pivot relative to each other and pivot/translate relative to platform 226, thereby extending each delivery assembly 220 outwardly (e.g., laterally) away from longitudinal axis 201. The outward extension of each delivery assembly 220 causes each delivery structure 128 to be positioned adjacent (or to engage with) the GI lumen wall for delivery of therapeutic preparation 125 (see, e.g., FIGS. 10A-13B and related discussion.) After delivery of therapeutic preparation 125 from delivery structures 128, the remaining portions of ingestible device 200 (e.g., delivery assembly 220, spring 210, support 215, delivery structure 128, and extender 230) can degrade and/or pass through the remainder of the GI tract and exit the anus of the subject.

As can be seen from the foregoing description, activation of a single release 208 causes actuator 210 to substantially simultaneously extend support 215 and extender 230 axially along longitudinal axis 201 (as indicated by a bidirectional arrow 236) and extend each delivery assembly 220 outwardly away from longitudinal axis 201 (as indicated by unidirectional arrows 238). Relying on a single release and actuator can allow for consistent actuation of support 215, delivery assemblies 220, and extender 230 so as to help to achieve consistent alignment and deployment of delivery structures 128 for delivery of therapeutic preparation 125. Additionally, using a single release reduces the number of components in ingestible device 200 thereby reducing assembly time and cost of the device. According to other embodiments, it may be advantageous to include multiple releases (in addition to release 208) in device 200 to cause sequential actuation of, for example, support 215, delivery assemblies 220, and extender 230.

For case of reference, certain components above (or below) support 215 are shown with reference numeral callouts in FIGS. 3A-3B whereas reference numeral callouts are not shown for similar components below (or above) support 215 (e.g., callouts for arms 222a-222d, pivot joints 224a-224h, platform 226, extender 230 and its ends 230a, 230b, frame members 232a-232h, pivot joints 234a-234i), but it should be appreciated that like components located above and below support 215 would be referenced by like callouts.

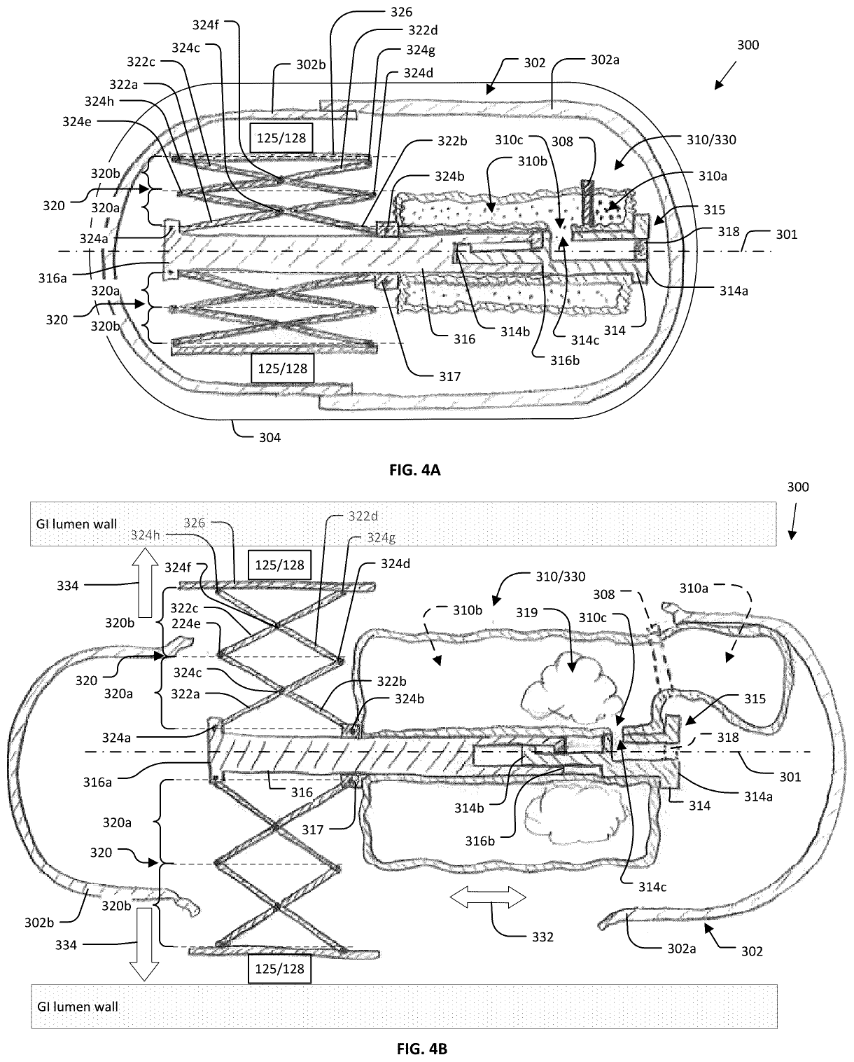

Referring now to FIG. 4A, a side cross-sectional view of an ingestible device 300 including an actuator in the form of an expandable member 310 (an embodiment of actuator 110) is shown. As discussed in greater detail below, expandable member 310 also functions as an extender 330 (an embodiment of extender 130) of the device. Ingestible device 300 is illustrated in a first state prior to ingestible device 300 reaching a desired location in the GI tract for delivering therapeutic preparation 125 into the GI lumen wall or surrounding tissue. Ingestible device 300 includes an enclosure 302 (an embodiment of enclosure 102) and an outer coating 304 (an embodiment of outer coating 104.) Enclosure 302 is structured as a capsule having a first segment 302a detachably coupled to a second segment 302b.

Ingestible device 300 further includes a support 315 (an embodiment of support 115) disposed within an interior of enclosure 302. Support 315 defines a generally longitudinal axis 301 of ingestible device 300. Support 315 includes a first support section 314 and a second support section 316. First support section 314 is movably coupled to second support section 316 so as to define a generally telescoping arrangement. In the example shown, first support section 314 is partially disposed within, and slidably coupled to, second support section 316. First support section 314 and second support section 316 have a complementary shape, or include portions that are complementary to each other to permit relative translatory (e.g., sliding) movement. First support section 314 defines a first support section first end 314a and a first support section second end 314b. Likewise, second support section 316 defines a second support section first end 316a and a second support section second end 316b. First support section 314 includes a channel 314c extending from an outer side portion of first support section 314 to first support section first end 314a. A release 318 is coupled to first support section first end 314a so as to temporarily block channel 314c. Channel 314c and release 318 collectively define a deflation valve for deflating expandable member 310, as discussed in greater detail below.

Still referring to FIG. 4A, expandable member 310 is coupled to first support section 314 at a first end and to a slidable member 317 at a second end located opposite the first end. Slidable member 317 is movably coupled to second support section 316 and is structured to translate (e.g., slide) axially relative to second support section 316 along longitudinal axis 301. Expandable member 310 is shown as an inflatable balloon, but expandable member 310 may be structured differently, such as another type of expandable structure having one or more concertinaed sides that can expand, similar to an accordion or bellows. Expandable member 310 substantially surrounds at least a portion of, or all of, support 315. At least a portion of expandable member 310 may be folded or otherwise arranged in the first state shown in FIG. 4A prior to expansion for an improved fit of expandable member 310 within the interior of enclosure 302.

Ingestible device 300 further includes a release 308 (an embodiment of release 108) coupled to expandable member 310. Release 308 is coupled to expandable member 310 in such a way so as to temporarily define a first chamber 310a and a second chamber 310b of expandable member 310. First chamber 310a is temporarily separated, and substantially sealed, from second chamber 310b. For example, release 308 may be located on an outer portion of expandable member 310 and may be structured as a band, a clip, or other structure to pinch, fold, or compress a portion (e.g., opposite side portions) of expandable member 310 so as to temporarily define separate first and second chambers 310a, 310b. First chamber 310a includes a first reactant contained therein, such as an acid (e.g., citric acid.) Second chamber 310b includes a second reactant contained therein, such as a carbonate (e.g., potassium bicarbonate.) Expandable member 310 is comprised of one or more layers of a flexible material (e.g., HPMC) to permit expansion of expandable member 310 in response to a chemical reaction caused by mixing of the first and second reactants within an interior of expandable member 310. Expandable member 310 further includes an opening 310c that is in fluid communication with channel 314c to function as part of the deflation valve for deflating expandable member 310.

Still referring to FIG. 4A, ingestible device 300 further includes a delivery assembly 320 (an embodiment of delivery assembly 120) pivotably coupled to support 315. In the embodiment shown in FIG. 4A, ingestible device 300 includes two delivery assemblies 320 positioned on opposite sides of support 315, although it should be appreciated that ingestible device 300 may include one or more, two or more, three or more, or four or more delivery assemblies 320 according to other embodiments. Delivery assembly 320 is structured similarly to delivery assembly 220 of FIGS. 3A-3B. Accordingly, for the sake of brevity, we refer to the description of delivery assembly 220 with respect to FIGS. 3A-3B for additional details regarding the structure and function of delivery assembly 320.

As shown in FIG. 4A, a plurality of arms 322a-322d pivotably couple each delivery assembly 320 to support 315 in a criss-cross manner by pivot joints 324a-324f to define first and second criss-cross sections 320a, 320b of a scissor mechanism (similar to a scissor lift) which, as previously discussed with reference to delivery assembly 220 in FIGS. 3A-3B, are structured to extend outwardly away from longitudinal axis 301 toward a lumen wall in the GI tract of a subject to deliver therapeutic preparation 125 into the GI lumen wall. Device 300 may include more, or fewer, than two criss-cross sections according to other embodiments. A first arm 322a is pivotably coupled to second support section 316 at or near second support section first end 316a. A second arm 322b is pivotably coupled to slidable member 317. Device 300 further includes a platform 326 pivotably and slidably coupled to second criss-cross section 320b by pivot joints 324g-324h. A delivery structure 128 containing one or more therapeutic preparations 125 therein is coupled to, or integrally formed with, platform 326.

Referring now to FIG. 4B, ingestible device 300 is shown in a second state when ingestible device 300 has reached a desired location in the GI tract of a subject to deliver therapeutic preparation 125 from delivery structure 128 into the GI lumen wall. As shown in FIG. 4B, enclosure 302 and/or outer coating 304 are partially (or fully) degraded as a result of ingestible device 300 having reached the desired location in the GI tract, which, for example, is associated with a selected or threshold pH value to trigger degradation of enclosure 302 and/or outer coating 304. As a result of at least partial degradation of enclosure 302 and/or outer coating 304, fluid in the GI tract is permitted to reach an interior of enclosure 302 to contact release 308 so as to activate (e.g., degrade) release 308. By activation of release 308, first and second chambers 310a, 310b become in fluidic communication which allows first reactant to mix with second reactant to cause a chemical reaction resulting in the formation of a gas 319 (e.g., carbon dioxide gas (CO2)) so as to cause expandable member 310 to expand outwardly away from, and axially along, longitudinal axis 301. Expansion of expandable member 310 along longitudinal axis 301 causes first support section 314 to move axially away from second support section 316 thereby extending an axial length of support 315 (as indicated by bidirectional arrow 332). Further, expansion of expandable member 310 causes slidable member 317 to translate (e.g., slide) axially along second support section 316 toward second support section first end 316a. For example, upon expansion of expandable member 310, the first end of expandable member 310 moves first support section 314 axially away from second support section 316. The second of expandable member 310 causes slidable member 317 to translate axially along second support section 316 toward second support section first end 316a.

As shown in FIG. 4B, the axial and/or outward expansion of expandable member 310 can, advantageously, help to push apart first and second segments 302a, 302b of capsule 302 to thereby assist in creating a substantially unobstructed area for deployment of delivery structures 128 toward the GI lumen wall. Moreover, expansion of expandable member 310 and the corresponding axial extension of support 315 helps to substantially align longitudinal axis 301 with a longitudinal axis of the GI lumen at the location in the GI lumen at which the expandable member 310 and support 315 are actuated. In this manner, expandable member 310 can effectively function as an extender 330 (an embodiment of extender 130) of ingestible device 300. Further, substantially aligning longitudinal axis 301 with the longitudinal axis of the GI lumen advantageously may help to orient delivery structures 128 to be substantially orthogonal relative to the GI lumen wall so as to facilitate delivery of therapeutic preparation 125 from delivery structure 128.

Axial movement of slidable member 317 along longitudinal axis 301 also causes the respective arms 322a-322d to pivot relative to each other such that each delivery assembly 320 extends outwardly away from longitudinal axis 301 toward the GI lumen wall (as indicated by unidirectional arrow 334.) For example, the translatory movement of second arm 322b toward first arm 322a via the slidable member 317 causes second arm 322b to pivot about second pivot joint 324b and first arm 322a to pivot about first pivot joint 324a, since first arm 322a is at a fixed position relative to support 315. This movement in turn causes third arm 322c and fourth arm 322d to pivot relative to each other and relative to platform 326 in a similar manner, thereby extending each delivery assembly 320 outwardly away from longitudinal axis 301. The outward extension of each delivery assembly 320 causes delivery structures 128 to be positioned adjacent (or to engage with) a GI lumen wall for delivery of therapeutic preparation 125 (see, e.g., FIGS. 10A-13B and related discussion.)

During and/or after delivery of therapeutic preparation 125 from delivery structure 128, the deflation valve is structured to allow gas 319 contained in expandable member 310 to pass through channel 314c and exit the device through first support section first end 314a such that expandable member 310 can deflate to help facilitate passage of device 300 through the remainder of the intestinal tract. For example, release 318 is coupled to first support section first end 314a so as to temporarily block channel 314c. Release 318 may be structured to degrade at a particular rate, or at a particular pH value to allow for sufficient time to enable expansion of expandable member 310 and extension of delivery assemblies 320 for delivery of therapeutic preparation 125 before releasing a substantial portion of gas 319 contained in the expandable member 310. Additionally or alternatively, release 318 may be structured to degrade, move, or open in response to a pressure within expandable member 310 (e.g., a threshold pressure value.) Additionally or alternatively, expandable member 310 may itself include a deflation valve, such as a flap or other structure that opens in response to an internal pressure within expandable member 310 so as to allow gas 319 to exit expandable member 310. The remaining portions of ingestible device 300 (e.g., delivery assembly 320, expandable member 310, support 315) can degrade and/or pass through the remainder of the GI tract and exit the anus of the subject.

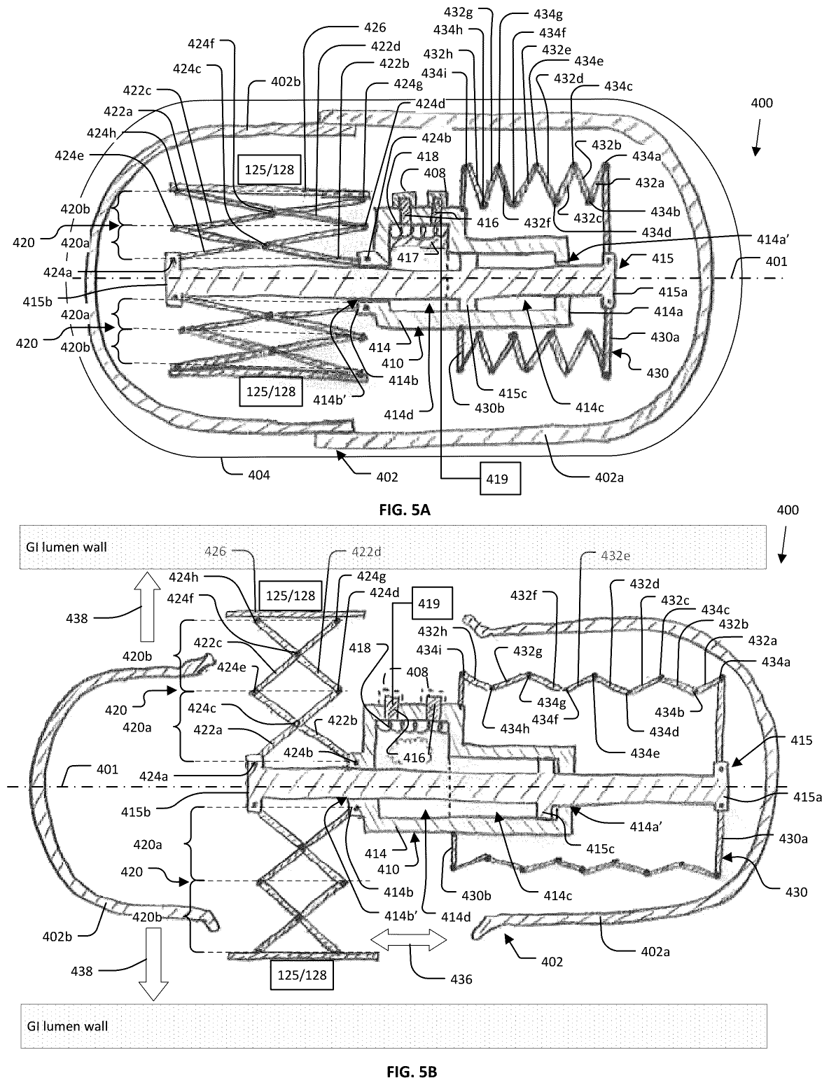

Referring now to FIG. 5A, a side cross-sectional view of an ingestible device 400 including an actuator 410 (an embodiment of actuator 110) in the form of a piston 415c slidably disposed in a housing 414 is shown. In the embodiment shown, actuator 410 further includes a charge 417 and an igniter 418 disposed in housing 414 for actuating piston 415c. In other embodiments (not shown), actuator 410 may include one or more reactants disposed in housing 414 for undergoing a chemical reaction to produce a gas (e.g., CO2) to actuate piston 415c. As shown in FIG. 5A, ingestible device 400 is illustrated in a first state prior to ingestible device 400 reaching a desired location in the GI tract for delivering therapeutic preparation 125 into the GI lumen wall or surrounding tissue thereof. Ingestible device 400 includes an enclosure 402 (an embodiment of enclosure 102) and an outer coating 404 (an embodiment of outer coating 104.) Enclosure 402 is structured as a capsule having a first segment 402a detachably coupled to a second segment 402b.

Ingestible device 400 further includes a support 415 (an embodiment of support 115) disposed within an interior of enclosure 402. Support 415 defines a generally longitudinal axis 401 of ingestible device 400. Support 415 includes a support first end 415a and a support second end 415b located opposite support first end 415a. Support 415 further includes piston 415c coupled to, or integrally formed with, support 415. Piston 415c is located between support first and second ends 415a, 415b. As discussed in greater detail below, support 415 is movable relative to housing 414 via piston 415c upon actuation of actuator 410.

Housing 414 defines a housing first end 414a having a housing first opening 414a′ and a housing second end 414b having a housing second opening 414b′. Housing 414 further defines an interior including a piston chamber 414c and a reaction chamber 414d. Piston chamber 414c is defined by an inner wall portion of housing 414 which extends axially along longitudinal axis 401. Reaction chamber 414d is defined by an inner wall portion of housing 414 positioned adjacent to piston chamber 414c. Reaction chamber 414d contains charge 417 and igniter 418 disposed therein. Charge 417 is positioned adjacent to and/or in contact with igniter 418. Charge 417 may be a combustible material, such as a composition comprising nitrocellulose (e.g., a nitrocellulose sheet) or other combustible material or combinations of materials. Igniter 418 may be a coil (as shown), a conductive wire or filament (e.g., tungsten filament), or other structure for generating heat, a spark, a flame, or other energy sufficient to ignite charge 417 in response to receiving an electrical signal (e.g., a current.)

Housing 414 further includes electrical contacts 416 coupled to a perimeter portion of housing 414. Electrical contacts 416 are operably coupled to igniter 418 and to an electrical circuit 419 (shown in block diagrammatic form in FIG. 5A.) Electrical circuit 419 may be located in housing 414. A release 408 (an embodiment of release 108) in the form of a coating or cover is applied to an outer portion of each of the electrical contacts 416 to temporarily prevent electrical contacts 416 from coming into contact with fluids in the GI tract in the first state shown in FIG. 5A. As discussed below, electrical circuit 419 is structured to generate a signal (e.g., a current) in response to a low resistance between electrical contacts 416 which occurs when fluid from the GI tract enters the space between electrical contacts 416 after release 408 no longer covers electrical contacts 416. Electrical circuit 419 is further structured such that igniter 418 initiates combustion of charge 417 in response to the generated signal.

For example, electrical circuit 419 may include a power source (e.g., a battery) and one or more energy storage components (e.g., a capacitor) coupled to housing 414. Electrical circuit 419 may further include one or more additional electrical components, such as a transistor, an electrical switch, a resistor, or other component. Electrical circuit 419 may be structured such that the electrical contacts 416 are shorted together by fluid in the GI tract, which allows for an electrical current from the power source to flow to the energy storage component. When a sufficient amount of current is accumulated in the energy storage component, the energy storage component can discharge the stored energy through the electrical circuit 419 across igniter 418 thereby causing igniter 418 to initiate combustion of charge 417.

Alternatively, electrical circuit 419 may include one or more sensors coupled to housing 414 for sensing a condition in the GI tract. Electrical circuit 419 may further include a processing circuit including a processor for executing instructions stored in a memory of the processing circuit in response to the sensed condition to cause igniter 418 to initiate combustion of charge 417. For example, the one or more sensors may be coupled to housing 414 and may be structured to sense a condition in the GI tract (e.g., a pH value, a temperature, an electrolytic value) to generate a corresponding signal. The processor can receive the signal and discharge the current stored in the energy storage component based on information stored in the memory of the processing circuit to thereby cause activation of igniter 418.

Still referring to FIG. 5A, support 415 is at least partially disposed within, and is movably coupled to, housing 414 such that piston 415c is slidably disposed within piston chamber 414c. Piston 415c may be slidably coupled to, and sealingly engaged with (e.g., via one or more piston rings, seals), an inner surface of housing 414. In the first state shown in FIG. 5A, support 415 extends through housing first opening 414a′ and housing second opening 414b′, such that support first end 415a is positioned outside of housing 414 adjacent to housing first end 414a and support second end 415b is positioned outside of housing 414 and housing second end 414b. Housing 414 may include one or more seals at housing first opening 414a′ and at housing second opening 414b′ for sealingly engaging with support 415 so as to create a substantially fluid-tight seal within piston chamber 414c and reaction chamber 414d. Housing 414, contacts 416, charge 417, igniter 418, and/or electrical circuit 419 may collectively define actuator 410. As discussed below, piston 415c moves axially within piston chamber 414c in response to ignition of charge 417 within reaction chamber 414d.

Still referring to FIG. 5A, ingestible device 400 further includes a delivery assembly 420 (an embodiment of delivery assembly 120) pivotably coupled to support 415. In the embodiment shown in FIG. 5A, ingestible device 400 includes two delivery assemblies 420 positioned on opposite sides of support 415, although it should be appreciated that ingestible device 400 may include one or more, two or more, three or more, or four or more delivery assemblies 420 according to other embodiments. Delivery assembly 420 is structured similarly to delivery assembly 220 of FIGS. 3A-3B. Accordingly, for the sake of brevity, we refer to the description of delivery assembly 220 with respect to FIGS. 3A-3B for additional details regarding the structure and function of delivery assembly 420.

As shown in FIG. 5A, device 400 further includes a plurality of arms 422a-422d pivotably coupled in a criss-cross manner by pivot joints 424a-424h to define first and second criss-cross sections 420a, 420b of a scissor mechanism (similar to a scissor lift) which, as previously discussed with reference to FIGS. 3A-3B, are structured to extend outwardly away from longitudinal axis 401 toward a lumen wall in the GI tract of a subject to deliver therapeutic preparation 125 into the GI lumen wall. Device 400 may include more or fewer than two criss-cross sections according to other embodiments. A second arm 422b is pivotably coupled to housing 414. A first arm 422a is pivotably coupled to support 415 at or near support second end 415b. Device 400 further includes a platform 426 pivotably and slidably coupled to an end portion of second criss-cross section 420b. A delivery structure 128 containing one or more therapeutic preparations 125 therein is coupled to, or integrally formed with, each platform 426.

Ingestible device 400 further includes an extender 430 (an embodiment of extender 130) coupled to support 415 and to housing 414. Extender 430 is structured to extend axially along longitudinal axis 401, so as to help orient ingestible device 400 within the GI lumen of a subject to facilitate delivery of therapeutic preparation 125 in a substantially perpendicular manner relative to the GI lumen wall. For example, extender 430 includes a plurality of frame members 432a-432h pivotably coupled together in a zig-zag pattern by pivot joints 434a-434i to define an expandable wire frame or skeleton-like structure. Extender 430 may define a generally elongated structure having any suitable cross-sectional shape (e.g., circular, triangular, pentagonal, hexagonal.) Additionally or alternatively, extender 430 may be a hollow, unitary structure comprising one or more concertinaed sides that can expand (similar to a bellows or an accordion.) Extender 430 defines an extender first end 430a and an extender second end 430b. Extender first end 430a is coupled to support first end 415a. Extender second end 430b is coupled to housing 414.

Referring now to FIG. 5B, ingestible device 400 is shown in a second state when ingestible device 400 has reached a desired location in the GI tract of a subject to deliver therapeutic preparation 125 from delivery structure 128 into the GI lumen wall of the subject. As shown in FIG. 5B, enclosure 402 and/or outer coating 404 are partially (or fully) degraded as a result of ingestible device 400 having reached the desired location in the GI tract, which, for example, is associated with a selected or threshold pH value to trigger degradation of enclosure 402 and/or outer coating 404. As a result of at least partial degradation of enclosure 402 and/or outer coating 404, fluid in the GI tract is permitted to reach an interior of enclosure 402 to contact release 408 so as to activate (e.g., degrade) release 408. In response to activation of release 408, electrical contacts 416 are exposed to bodily fluid in the GI tract to cause an electrical short across electrical contacts 416 so as to allow a flow of current through electrical circuit 419. The current generated from electrical circuit 419 is sufficient to cause igniter 418 to ignite charge 417 to generate a combustion force within reaction chamber 414d. The force generated in reaction chamber 414d causes piston 415c and thus support 415 to move axially along longitudinal axis 401 toward housing first end 414a (as indicated by bidirectional arrow 436.) This axial movement of support 415 causes extender 430 to extend axially away from housing 414 (as indicated by bidirectional arrow 436), since extender first end 430a is at a fixed axial position relative to support first end 415a and extender second end 430b is coupled to housing 414.

As shown in FIG. 5B, the axial extension of extender 430 can, advantageously, help to push apart first and second segments 402a, 402b of capsule 402 to thereby assist in creating a substantially unobstructed area for deployment of delivery structures 128 toward the GI lumen wall. Moreover, axial extension of extender 430 helps to substantially align longitudinal axis 401 with a longitudinal axis of the GI lumen at the location in the GI lumen at which the extender 430 is actuated. Substantially aligning longitudinal axis 401 with the longitudinal axis of the GI lumen can help to orient delivery structures 128 to be substantially orthogonal relative to the GI lumen wall so as to facilitate delivery of therapeutic preparation 125 from delivery structure 128.

Axial movement of support 415 along longitudinal axis 401 relative to housing 414 also causes the respective arms 422a-422d to pivot relative to each other such that each delivery assembly 420 extends outwardly away from longitudinal axis 401 toward the GI lumen wall (as indicated by unidirectional arrow 438.) For example, the translatory movement of support 415 along longitudinal axis 401 relative to housing 414 causes second arm 422b to pivot about second pivot joint 424b and first arm 422a to pivot about first pivot joint 424a, since first arm 422a is at a fixed position on support 415 and second arm 422b is coupled to housing 414. This in turn causes third arm 422c and fourth arm 422d to pivot relative to each other and relative to platform 426 in a similar manner, thereby extending each delivery assembly 420 outwardly away from longitudinal axis 401. The outward extension of each delivery assembly 420 causes delivery structures 128 to be positioned adjacent (or to engage with) a GI lumen wall for delivery of therapeutic preparation 125 into the lumen wall (see, e.g., FIGS. 10A-13B and related discussion.) After delivery of therapeutic preparation 125 from delivery structure 128, the remaining portions of ingestible device 400 (e.g., one or more of delivery assembly 420, housing 414, support 415, and extender 430) can degrade and/or pass through the remainder of the GI tract and exit the anus of the subject.

For ease of reference, certain components above (or below) support 415 are shown with reference numeral callouts in FIGS. 5A-5B whereas reference numeral callouts are not shown for similar components below (or above) support 415, but it should be appreciated that like components located above and below support 415 would be referenced by like callouts.

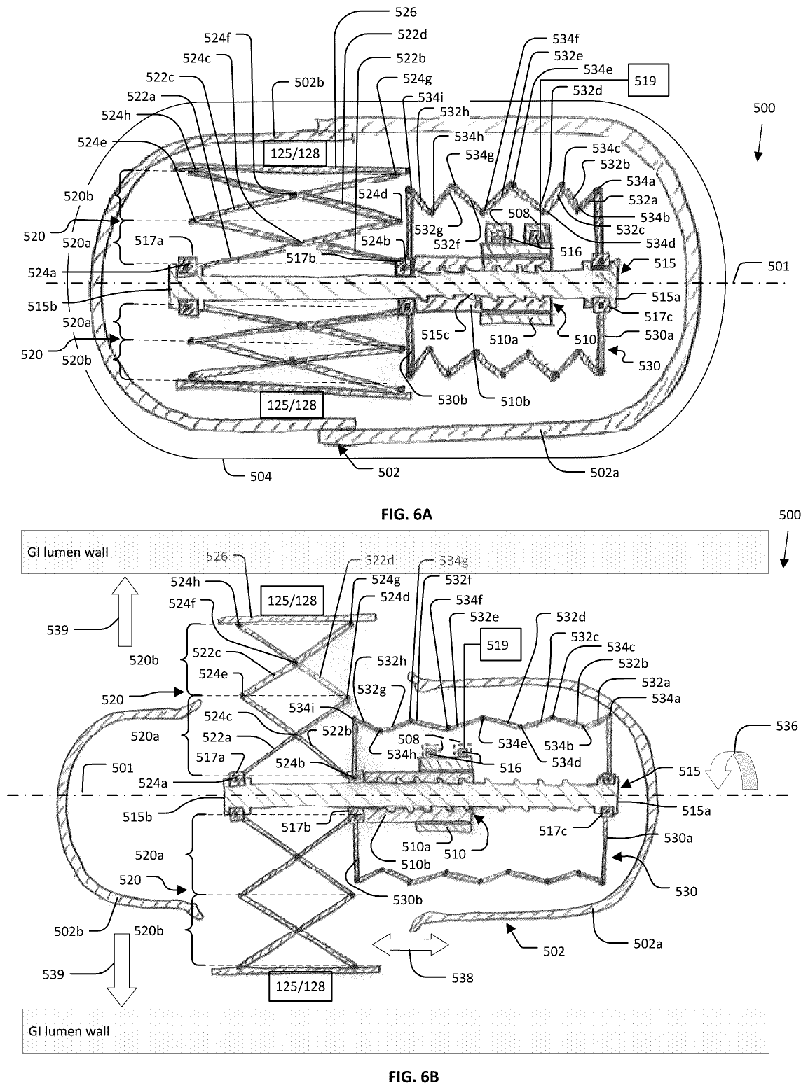

Referring to FIG. 6A, a side cross-sectional view of an ingestible device 500 including an actuator in the form of a motor 510 (an embodiment of actuator 110) is shown. In the embodiment shown, motor 510 is a screw-type rotary motor. In other embodiments (not shown), motor 510 is a linear motor. As shown in FIG. 6A, ingestible device 500 is illustrated in a first state prior to ingestible device 500 reaching a desired location in the GI tract for delivering therapeutic preparation 125 into the GI lumen wall. Ingestible device 500 includes an enclosure 502 (an embodiment of enclosure 102) and an outer coating 504 (an embodiment of outer coating 104.) Enclosure 502 is structured as a capsule having a first segment 502a detachably coupled to a second segment 502b.

Ingestible device 500 further includes a support 515 (an embodiment of support 115) disposed within an interior of enclosure 502. Support 515 defines a generally longitudinal axis 501 of ingestible device 500. Support 515 includes a support first end 515a and a support second end 515b located opposite support first end 515a. Support 515 further includes an outer threaded portion 515c located between first and second ends 515a, 515b. Threaded portion 515c may extend at least a portion of, or along the entire length of, support 515. Threaded portion 515c is threadably coupled to motor 510 such that support 515 extends through motor 510 and is movable relative to motor 510. As discussed in greater detail below, support 515 is structured to translate along longitudinal axis 501 via rotational movement of threaded portion 515c relative to motor 510 upon actuation of motor 510. In other embodiments (not shown), support 515 includes a linear gear that functions as a rack and motor 510 includes a rotary gear that functions as a pinion which is rotatably coupled to the linear gear for translating support 515 relative to motor 510 along longitudinal axis 501.

Still referring to FIG. 6A, motor 510 is a screw-type rotary motor including a motor housing 510a and a rotatable shaft 510b. Motor 510 may be an electric motor (e.g., a micro direct current (DC) motor, a synchronous or asynchronous motor, a micro alternating current (AC) motor, a stepper motor, or a servomotor), a nano motor, a magnetic motor, or any other type of micro-scale rotary motor that can be contained within capsule 502. Rotatable shaft 510b is rotatable relative to motor housing 510a upon actuation of motor 510. Rotatable shaft 510b is hollow and includes an inner threaded portion that is complementary to outer threaded portion 515c of support 515 such that support 515 can be threadably coupled to rotatable shaft 510b. Motor housing 510a includes electrical contacts 516 and an electrical circuit 519 (shown in block diagrammatic form) operatively coupled to electrical contacts 516. A release 508 (an embodiment of release 108) in the form of a coating, or cover is disposed on electrical contacts 516 to temporarily prevent contact with fluid in the GI tract in the first state shown in FIG. 6A. For example, release 508 may be a degradable coating or layer of material that substantially covers an outer portion of electrical contacts 516. Electrical circuit 519 is structured to generate a signal (e.g., a current) to operate motor 510 in response to an electrical short between electrical contacts 516 based on contact with bodily fluid in the GI tract when release 508 is activated (e.g., degraded.) For example, electrical circuit 519 may include a power source (e.g., a battery) coupled to motor 510 and one or more additional electrical components for providing a current to operate motor 510 in response to the electrical short so as to cause rotatable shaft 510b to rotate.

Still referring to FIG. 6A, ingestible device 500 further includes a delivery assembly 520 (an embodiment of delivery assembly 120) pivotably coupled to support 515. In the embodiment shown in FIG. 6A, ingestible device 500 includes two delivery assemblies 520 positioned on opposite sides of support 515, although it should be appreciated that ingestible device 500 may include one or more, two or more, three or more, or four or more delivery assemblies 520 according to other embodiments. Delivery assembly 520 is structured identically to delivery assembly 220 of FIGS. 3A-3B. Accordingly, for the sake of brevity, we refer to the description of delivery assembly 220 with respect to FIGS. 3A-3B for additional details regarding the structure and function of delivery assembly 520.

As shown in FIG. 6A, device 500 further includes a plurality of arms 522a-522d pivotably coupled together in a criss-cross manner by respective pivot joints 524a-524h to define first and second criss-cross sections 520a, 520b of a scissor mechanism (similar to a scissor lift) which, as previously discussed with reference to FIGS. 3A-3B, are structured to extend outwardly away from longitudinal axis 501 toward a lumen wall in the GI tract of a subject to deliver therapeutic preparation 125. Device 500 may include more or fewer than two criss-cross sections according to other embodiments. A first arm 522a is pivotably coupled to a first rotatable member 517a by a first pivot joint 524a. First rotatable member 517a is rotatably coupled to support 515, but is fixed axially relative to support 515 at or near support second end 515b. A second arm 422b is pivotably coupled to support 515 via a second rotatable member 517b. Second rotatable member 517b is movably coupled (e.g., slidable) to support 515 and is structured such that support 515 can translate and rotate relative to second rotatable member 517b. First rotatable member 517a and second rotatable member 517b may be structured as a bushing, a rotary bearing, a sleeve, or similar component. Device 500 further includes a platform 526 pivotably and slidably coupled to an end portion of second criss-cross section 520b. A delivery structure 128 containing one or more therapeutic preparations 125 therein is coupled to, or integrally formed with, each platform 526.

Ingestible device 500 further includes an extender 530 (an embodiment of extender 130) coupled to support 515 and to motor housing 510a. Extender 530 is structured to extend axially along longitudinal axis 501, so as to help orient ingestible device 500 within the GI lumen of a subject to facilitate delivery of therapeutic preparation 125 in a substantially perpendicular manner relative to the GI lumen wall. For example, extender 530 includes a plurality of linear frame members 532a-532h pivotably coupled together in a zig-zag pattern by respective pivot joints 534a-534i to define an expandable wire frame or skeleton-like structure. Extender 230 may define a generally elongated structure having any suitable cross-sectional shape (e.g., circular, triangular, pentagonal, hexagonal.) Additionally or alternatively, extender 530 may be a hollow, unitary structure comprising one or more concertinaed sides that can expand (similar to a bellows or an accordion.) Extender 530 defines an extender first end 530a and an extender second end 530b. Extender first end 530a is coupled to a third rotatable member 517c, which is rotatably coupled to support 515 at or near support first end 515a. Third rotatable member 517c may be structured similarly as first and second rotatable members 517a, 517b. Extender second end 530b is coupled to second rotatable member 517b (e.g., via second pivot joint 524b.) Extender 530 is shown disposed over release 508 and motor 510, however, it should be appreciated that release 508 may be accessible through one or more openings of extender 530 to permit activation of release 508 (e.g., degradation resulting from contact with fluid in the GI tract.)

Referring now to FIG. 6B, ingestible device 500 is shown in a second state when ingestible device 500 has reached a desired location in the GI tract of a subject to deliver therapeutic preparation 125 from delivery structure 128 into the GI lumen wall of the subject. As shown in FIG. 6B, enclosure 502 and/or outer coating 504 are partially (or fully) degraded as a result of ingestible device 500 having reached the desired location in the GI tract, which, for example, is associated with a selected or threshold pH value to trigger degradation of enclosure 502 and/or outer coating 504. As a result of at least partial degradation of enclosure 502 and/or outer coating 504, fluid in the GI tract is permitted to reach an interior of enclosure 502 to contact release 508 so as to activate (e.g., degrade) release 508. In response to activation of release 508, fluid in the GI tract creates an electrical short across electrical contacts 516 so as to allow an electrical current to flow through electrical circuit 519. The current from electrical circuit 519 is sufficient to operate motor 510 to generate a rotational force to rotate rotatable shaft 510a about longitudinal axis 501 (as indicated by rotational arrow 536.) This rotational motion of rotatable shaft 510b causes support 515 to rotate about, and move axially along, longitudinal axis 401 via threaded engagement with threaded portion 515. This axial movement of support 515 causes extender 530 to extend axially away from motor 510, since extender first end 530a is at a fixed axial position relative to motor housing 510a and extender second end 530b is rotatably coupled to support first end 515a at a fixed axial position on support 515.

As shown in FIG. 6B, the axial extension of extender 530 can, advantageously, help to push apart first and second segments 502a, 502b of capsule 502 (as indicated by bidirectional arrow 538) to thereby assist in creating a substantially unobstructed area for deployment of delivery structures 128 toward the GI lumen wall. Moreover, axial extension of extender 530 may help to substantially align longitudinal axis 501 with a longitudinal axis of the GI lumen at the location in the GI lumen at which the extender 530 is actuated. Substantially aligning longitudinal axis 501 with the longitudinal axis of the GI lumen can advantageously help to orient delivery structures 128 to be substantially orthogonal relative to the GI lumen wall so as to facilitate delivery of therapeutic preparation 125 from delivery structure 128 into the lumen wall.

Axial movement of support 515 along longitudinal axis 501 relative to motor 510 also causes the respective arms 522a-522d to pivot relative to each other such that each delivery assembly 520 extends outwardly away from longitudinal axis 501 toward the GI lumen wall (as indicated by unidirectional arrow 539.) For example, the translatory movement of support 515 along longitudinal axis 501 relative to motor 510 causes second arm 522b to pivot about second pivot joint 524b and first arm 522a to pivot about first pivot joint 524a, since second arm 522b can translate relative to support 515 via second rotatable member 517b and first arm 522a is fixed axially relative to support 515 via first rotatable member 517a. This in turn causes third arm 522c and fourth arm 522d to pivot relative to each other and relative to platform 526 in a similar manner, thereby extending each delivery assembly 520 outwardly away from longitudinal axis 501. The outward extension of each delivery assembly 520 causes delivery structures 528 to be positioned adjacent (or to engage with) a GI lumen wall for delivery of therapeutic preparation 125 into the lumen wall (see, e.g., FIGS. 10A-13B and related discussion.) After delivery of therapeutic preparation 125 from delivery structure 128, the remaining portions of ingestible device 500 (e.g., one or more of delivery assembly 520, motor 510, support 515, and extender 530) can degrade and/or pass through the remainder of the GI tract and exit the anus of the subject.

For ease of reference, certain components above (or below) support 515 are shown with reference numeral callouts in FIGS. 6A-6B whereas reference numeral callouts are not shown for similar components below (or above) support 515, but it should be appreciated that like components located above and below support 515 would be referenced by like callouts.

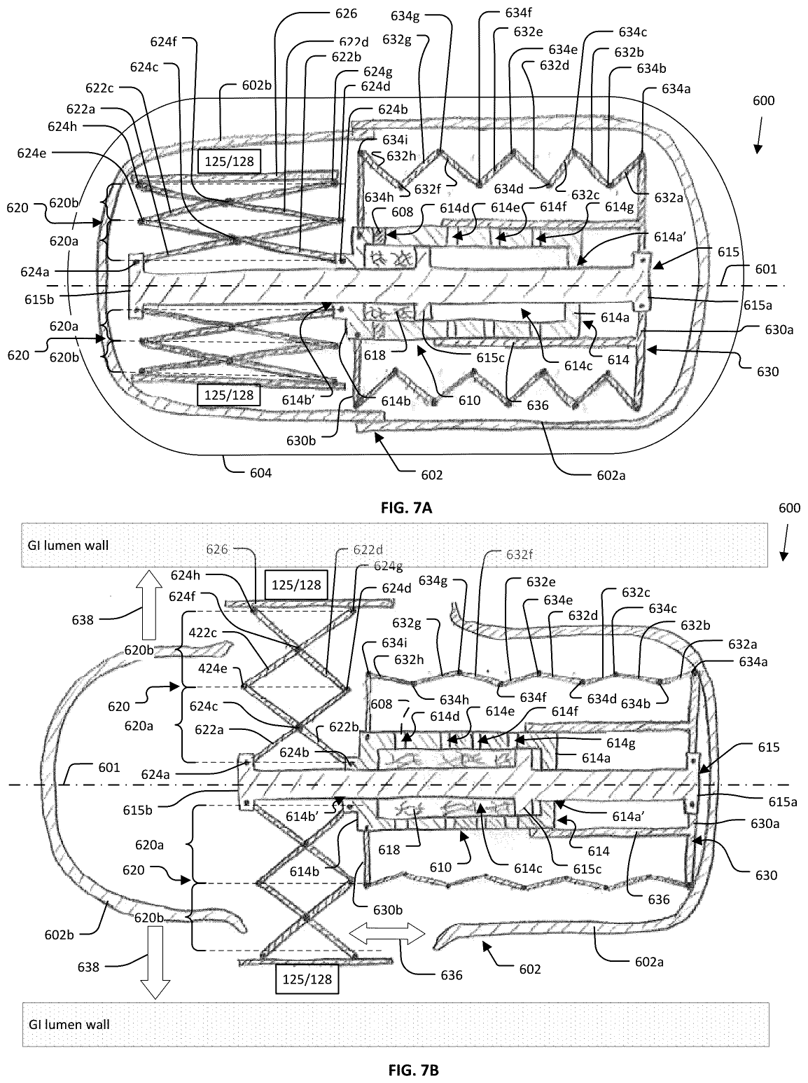

Referring now to FIG. 7A, a side cross-sectional view of an ingestible device 600 including an actuator 610 (an embodiment of actuator 110) in the form of a piston 615c slidably disposed in a housing 614 is shown. In the embodiment shown, actuator 610 further includes a hydrogel 618 disposed in housing 614 for actuating piston 615c. As shown in FIG. 7A, ingestible device 600 is illustrated in a first state prior to ingestible device 600 reaching a desired location in the GI tract for delivering therapeutic preparation 125 into the GI lumen wall. Ingestible device 600 includes an enclosure 602 (an embodiment of enclosure 102) and an outer coating 604 (an embodiment of outer coating 104.) Enclosure 602 is structured as a capsule having a first segment 602a detachably coupled to a second segment 602b.

Ingestible device 600 further includes a support 615 (an embodiment of support 115) disposed within an interior of enclosure 602. Support 615 defines a generally longitudinal axis 601 of ingestible device 600. Support 615 includes a support first end 615a and a support second end 615b located opposite support first end 615a. Support 615 further includes piston 615c coupled to, or integrally formed with, support 615. Piston 615c is located between support first and second ends 615a, 615b. As discussed in greater detail below, support 615 is movable relative to housing 614 via piston 615c upon actuation of actuator 610.

Housing 614 which may form part of actuator 610. Housing 614 defines a housing first end 614a having a housing first opening 614a′ and a housing second end 614b having a housing second opening 614b′. Housing 614 further defines an interior including a piston chamber 614c. Piston chamber 614c is defined by an inner surface of housing 614 which extends axially along longitudinal axis 601. Hydrogel 618 is positioned adjacent to and/or in contact with piston 615c in front of piston 615c near housing second opening 614b within piston chamber 614c. In the embodiment shown, hydrogel 618 is a three-dimensional polymer that is structured to swell or expand within piston chamber 614c in response to a stimuli in the GI tract, such as fluid in the GI tract having a threshold pH value, or range of pH values associated with a particular location in the GI tract for delivery of therapeutic preparation 125. For example, hydrogel 618 may comprise one or more polyelectrolytes that are pH-responsive. Additionally or alternatively, hydrogel 618 may include one or more polymers that are responsive to other types of stimuli, such as temperature, light, ionic strength, or other stimuli associated with a particular location in the GI tract (e.g., the small intestine.) Hydrogel 618 may be structured to bend, deform, or otherwise move so as to cause axial movement of piston 615c within piston chamber 614c. Hydrogel 618 may be in the form of a film, a gel, or other physical structure having various shapes and configurations which can be disposed in piston chamber 614c.

Housing 614 further includes a plurality of openings 614d-614g located along a perimeter of housing 614. Openings 614d-614g are spaced apart from each other and are distributed along an axial length of housing 614. Openings 614d-614g are structured to provide a fluid path from the GI lumen environment to piston chamber 614c to cause progressive actuation of hydrogel 618. In the first state shown in FIG. 7A, a first pair of openings 614d located closest to housing second end 614b adjacent to hydrogel 618 are temporarily blocked by a release 608 (an embodiment of release 108.) Release 608 may be a degradable release structured as a plug or similar structure for temporarily blocking the first pair of openings 618d. As discussed below, upon activation of release 608 (e.g., degradation from contact with fluids in the GI tract), fluid from the GI lumen environment is permitted to enter piston chamber 614c to contact hydrogel 618 to cause hydrogel 618 to expand and thereby cause piston 615c to move axially along longitudinal axis 601 toward housing first end 614a. As discussed in greater detail below, as piston 615c moves axially toward first end 614a due to initial expansion of hydrogel 618, a cover 636 coupled to or integrally formed with an extender 630 (an embodiment of extender 130) is moved away from housing 414 to progressively expose openings 614e-614g to allow additional fluid from the GI lumen environment to contact hydrogel 618 and thereby cause progressive actuation of hydrogel 618 within piston chamber 614c. Alternatively, cover 636 may be coupled to support 615.

Still referring to FIG. 7A, support 615 is disposed within, and is movably coupled to, housing 614 such that piston 615c is slidably disposed within piston chamber 614c. Piston 615c is located in front of hydrogel 618 near housing second end 614b. Piston 615c may be slidably coupled to, and sealingly engaged with (e.g., via one or more piston rings, seals), an inner surface of housing 414. Support 615 extends through housing first opening 614a and housing second opening 614b, such that support first end 615a is positioned outside of housing 614 adjacent to housing first end 614a and support second end 615a is positioned outside of housing 614 near housing second end 614b in the first state shown in FIG. 7A. Housing 614 may include one or more seals at housing first opening 614a′ and housing second opening 614b′ for sealingly engaging with support 615 to create a substantially fluid tight seal within piston chamber 614c. Housing 614, piston 615c, and/or hydrogel 618 may collectively define actuator 610.

Still referring to FIG. 7A, ingestible device 600 further includes a delivery assembly 620 (an embodiment of delivery assembly 120) pivotably coupled to support 615 and housing 614. In the embodiment shown in FIG. 7A, ingestible device 600 includes two delivery assemblies 620 positioned on opposite sides of support 615, although it should be appreciated that ingestible device 600 may include one or more, two or more, three or more, or four or more delivery assemblies 620 according to other embodiments. Delivery assembly 620 is structured similarly to delivery assembly 220 of FIGS. 3A-3B. Accordingly, for the sake of brevity, we refer to the description of delivery assembly 220 with respect to FIGS. 3A-3B for additional details regarding the structure and function of delivery assembly 620.

As shown in FIG. 7A, device 600 further includes a plurality of arms 622a-622d pivotably coupled together in a criss-cross manner by respective pivot joints 624a-624h to define first and second criss-cross sections 620a, 620b of a scissor mechanism (similar to a scissor lift) which, as previously discussed with reference to FIGS. 3A-3B, are structured to extend outwardly away from longitudinal axis 601 toward a lumen wall in the GI tract of a subject to deliver therapeutic preparation 125 into the GI lumen wall. Device 600 may include more or fewer than two criss-cross sections according to other embodiments. A first arm 622a is pivotably coupled to support 615 at or near support second end 615b. A second arm 622b is pivotably coupled to housing 614 at or near housing second end 614b. Device 600 further includes a platform 626 pivotably and slidably coupled to an end portion of second criss-cross section 620b. A delivery structure 628 containing one or more therapeutic preparations 125 therein is coupled to, or integrally formed with, each platform 626.

Ingestible device 600 further includes an extender 630 (an embodiment of extender 130) coupled to support 615 and to housing 614. Extender 630 is structured to extend axially along longitudinal axis 601, so as to help orient ingestible device 600 within the GI lumen of a subject which can help to facilitate delivery of therapeutic preparation 125 in a substantially perpendicular manner relative to the GI lumen wall. For example, extender 630 includes a plurality of linear frame members 632a-632h pivotably coupled together in a zig-zag pattern by respective pivot joints 634a-634i (e.g., pin, ball and socket joint, spring joint) to define an expandable wire frame or skeleton-like structure. Extender 630 may define a generally elongated structure having any suitable cross-sectional shape (e.g., circular, triangular, pentagonal, hexagonal.) Additionally or alternatively, extender 630 may be a hollow, unitary structure comprising one or more concertinaed sides that can expand (similar to a bellows or an accordion.) Extender 630 defines an extender first end 630a and an extender second end 630b. Extender first end 630a is coupled to housing 614. Extender second end 630b is coupled to support first end 615a.

A cover 636 is coupled to, or integrally formed with, extender 630. Cover 636 is disposed over a portion of housing 614 such that cover 636 temporarily blocks openings 614c-614g to prevent fluid from the GI lumen environment to enter piston chamber 614c. Cover 636 may be structured as a sleeve or other type of covering. Cover 636 is structured to move relative to housing 614 in response to axial movement of extender 630 so as to selectively expose openings 614c-614g and allow fluid from the GI lumen environment to enter piston chamber 614c. Additionally or alternatively, cover 636 may be coupled to support 615 such that axial movement of support 615 directly causes cover 636 to move relative to housing 614. In this manner, cover 636 can allow for progressive actuation of hydrogel 618.

Referring now to FIG. 7B, ingestible device 600 is shown in a second state when ingestible device 600 has reached a desired location in the GI tract of a subject to deliver therapeutic preparation 125 from delivery structure 128 into the GI lumen wall of the subject. As shown in FIG. 7B, enclosure 602 and/or outer coating 604 are partially (or fully) degraded as a result of ingestible device 600 having reached the desired location in the GI tract, which, for example, is associated with a selected or threshold pH value to trigger degradation of enclosure 602 and/or outer coating 604. As a result of at least partial degradation of enclosure 602 and/or outer coating 604, fluid in the GI tract is permitted to reach an interior of enclosure 602 to contact release 608 so as to activate (e.g., degrade) release 608. In response to activation of release 608, fluid in the GI tract is permitted to enter piston chamber 614 via first openings 614d located adjacent to hydrogel 618 so as to cause initial expansion or swelling of hydrogel 618. Expansion of hydrogel 618 causes piston 615c to move axially along longitudinal axis 601 toward housing first end 614a, thereby causing support 615 to move axially in the same direction relative to housing 614. As piston 615c moves axially toward housing first end 614a, extender 630 extends axially and cover 636 is moved away from housing 614 to expose additional openings 614c-614g on housing 614 to allow additional fluid from the GI lumen environment to enter piston chamber 614c and contact hydrogel 618. The additional fluid entering piston chamber 614c causes progressive expansion of hydrogel 618 and subsequent axial movement of support 615.

As shown in FIG. 7B, the axial extension of extender 630 can, advantageously, help to push apart first and second segments 602a, 602b of capsule 602 (as indicated by bidirectional arrow 636) to thereby assist in creating a substantially unobstructed area for deployment of delivery structures 628 toward the GI lumen wall. Moreover, axial extension of extender 630 can help substantially align longitudinal axis 601 with a longitudinal axis of the GI lumen at the location in the GI lumen at which the extender 630 is actuated. Substantially aligning longitudinal axis 601 with the longitudinal axis of the GI lumen may help to orient delivery assemblies 620 to be substantially orthogonal relative to the GI lumen wall which can facilitate delivery of therapeutic preparation 125 from delivery structure 628.

Axial movement of support 615 along longitudinal axis 601 relative to housing 614 also causes the respective arms 622a-622d to pivot relative to each other such that each delivery assembly 620 extends outwardly away from longitudinal axis 601 toward the GI lumen wall (as indicated by unidirectional arrow 638.) For example, the axial movement of support 615 along longitudinal axis 601 relative to housing 614 causes second arm 622b to pivot about second pivot joint 624b and first arm 622a to pivot about first pivot joint 624a, since first arm 422a is coupled to movable support 615 and second arm 622b is at a fixed axial position on housing 614. This in turn causes third arm 622c and fourth arm 622d to pivot relative to each other and relative to platform 626 in a similar manner, thereby extending each delivery assembly 620 outwardly away from longitudinal axis 601. The outward extension of each delivery assembly 620 causes delivery structures 628 to be positioned adjacent (or to engage with) a GI lumen wall for delivery of therapeutic preparation 125 (see, e.g., FIGS. 10A-13B and related discussion.) After delivery of therapeutic preparation 125 from delivery structure 628, the remaining portions of ingestible device 600 (e.g., one or more of delivery assembly 620, housing 614, support 615, and extender 630) can degrade and/or pass through the remainder of the GI tract and exit the anus of the subject.

For ease of reference, certain components above (or below) support 615 are shown with reference numeral callouts in FIGS. 7A-7B whereas reference numeral callouts are not shown for similar components below (or above) support 615, but it should be appreciated that like components located above and below support 615 would be referenced by like callouts.

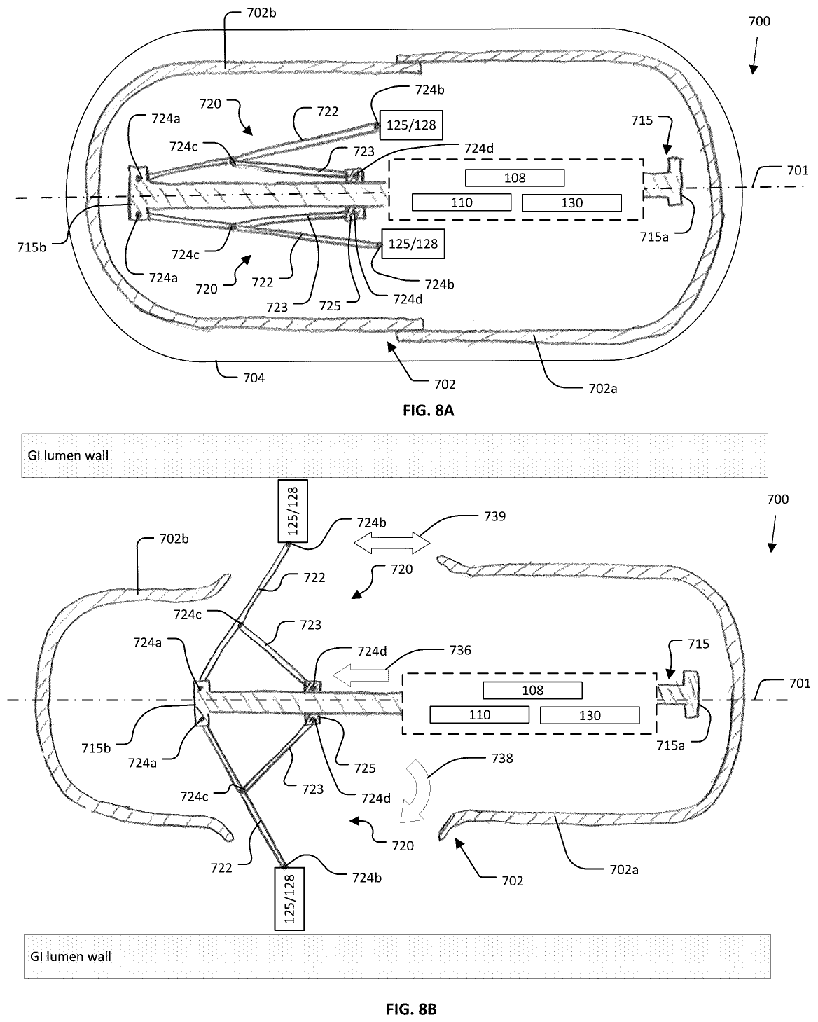

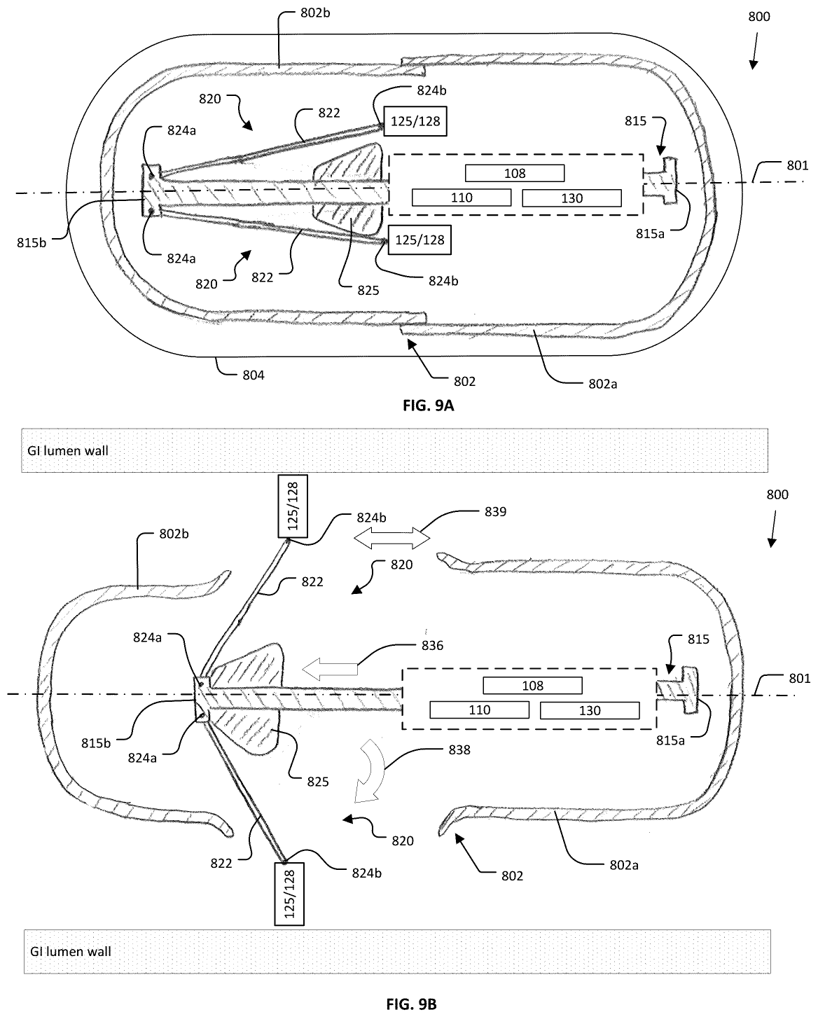

Referring generally to FIGS. 8A-9B, additional or alternative embodiments of delivery assembly 120 are shown in the form of delivery assemblies 720, 820. Delivery assemblies 720, 820 are structured to be coupled to a support via a pivotable arm structured to pivot relative to the support about a pivot axis defined by a pivot joint (e.g., a pin, a hinge, a ball and socket, a spring joint, or other pivot joint) on the support, such that the pivotable arm extends outwardly away from the support toward a GI lumen wall (similar to the function of an umbrella.) Delivery assemblies 720, 820 may be used in any of the preceding embodiments in conjunction with any one of, or a combination of, the releases, actuators, and/or extenders previously described with reference to FIGS. 3A-7B. As such, the corresponding release 108, actuator 110, and extender 130 of each device are depicted in block diagrammatic form in FIGS. 8A-9B.

Referring now to FIG. 8A, a side cross-sectional view of an ingestible device 700 including a delivery assembly 720 (an embodiment of delivery assembly 120) in the form of a pivotable arm 722 is shown. As shown in FIG. 8A, ingestible device 700 is illustrated in a first state prior to ingestible device 700 reaching a desired location in the GI tract for delivering therapeutic preparation 125 into the GI lumen wall. Ingestible device 700 further includes an enclosure 702 (an embodiment of enclosure 102) and an outer coating 704 (an embodiment of outer coating 104.) Enclosure 702 is structured as a capsule having a first segment 702a detachably coupled to a second segment 702b.

Ingestible device 700 further includes a support 715 (an embodiment of support 115). Support 715 is a generally elongated member that defines a generally longitudinal axis 701 of ingestible device 700. Support 715 includes a support first end 715a and a support second end 715b located opposite support first end 715a. Support 715 may have any one of, or a combination of, the structural configurations of support 115 previously described with reference to the embodiments of FIGS. 3A-7B.

Ingestible device 700 further includes a delivery assembly 720 (an embodiment of delivery assembly 120) pivotably coupled to support 715. In the embodiment shown in FIG. 8A, ingestible device 700 includes two delivery assemblies 720 positioned on opposite sides of support 715, although it should be appreciated that ingestible device 700 may include one or more, two or more, three or more, or four or more delivery assemblies 720 according to other embodiments. Each delivery assembly 720 includes a pivotable arm 722. Pivotable arm 722 is shown as a substantially linear member, but pivotable arm 722 may be structured to be substantially non-linear or include substantially non-linear portions, according to other embodiments. A first end of pivotable arm 722 is pivotably coupled to support 715 at or near support second 715b via a first pivot joint 724a. A second, opposite end of pivotable arm 722 is pivotably coupled to a delivery structure 728 containing one or more therapeutic preparations 125 therein by a second pivot joint 724b. Delivery structure 728 is shown in block diagrammatic form in FIGS. 8A-8B, but various embodiments of delivery structure 728 are discussed below with reference to FIGS. 10A-13B.

Still referring to FIG. 8A, each delivery assembly 720 further includes a rib 723 pivotably coupled to pivotable arm 722 at a first end via third pivot joint 724c between the two ends of pivotable arm 722. Rib 723 is a substantially linear member, but rib 723 may be structured to be substantially non-linear or include substantially non-linear portions, according to other embodiments. Rib 723 includes an opposite, second end pivotably coupled to a slidable member 725 by a fourth pivot joint 724d. The various pivot joints 724a-724d may be structured similarly as any of the other pivot joints previously described herein. Slidable member 725 may be a hub, a sleeve, a bushing, or other similar structure that can translate (e.g., slide) relative to support 715 along longitudinal axis 701. Ingestible device 700 is structured such that actuation of actuator 710 causes slidable member 725 to translate axially along longitudinal axis 701 relative to support 715 toward support second end 715b to thereby cause each delivery assembly 720 to pivot about first pivot axis 724a and extend outwardly away from support 715 toward a GI lumen wall for delivery of therapeutic preparation 125 from delivery structure 128.

For example, referring to FIG. 8B, ingestible device 700 is shown in a second state when ingestible device 700 has reached a desired location in the GI tract of a subject to deliver therapeutic preparation 125 from delivery structure 128 into the GI lumen wall. As shown in FIG. 8B, enclosure 702 and/or outer coating 704 are partially (or fully) degraded as a result of ingestible device 700 having reached the desired location in the GI tract, which, for example, is associated with a selected or threshold pH value to trigger degradation of enclosure 702 and/or outer coating 704. As a result of at least partial degradation of enclosure 702 and/or outer coating 704, fluid in the GI tract is permitted to reach an interior of enclosure 702 to contact release 108 so as to activate (e.g., degrade) release 108. In response to activation of release 108, actuator 110 is actuated to cause extender 130 to extend axially along longitudinal axis 701 and to cause slidable member 725 to translate axially along longitudinal axis 701 toward support second end 715b (as indicated by unidirectional arrow 736.) This axial movement of slidable member 725 causes each of the ribs 723 to pivot relative to slidable member 725 and relative to the respective pivotable arms 722 to thereby cause pivotable arms 722 to pivot about first pivot joint 724a (as indicated by rotational arrow 738) and extend outwardly away from support 115, similar to the function of an umbrella. The outward movement of pivotable arms 722 causes delivery structures 728 to be positioned adjacent (or in contact with) the GI lumen wall to permit delivery of therapeutic preparation 125 into the lumen wall (see, e.g., FIGS. 10A-13B and related discussion.) Further, the outward movement of pivotable arms 722 can assist with the separation of first and second segments 702a, 702b of capsule 702 (as indicated by bidirectional arrow 739.)

Referring now to FIG. 9A, a side cross-sectional view of an ingestible device 800 including a delivery assembly 820 (an embodiment of delivery assembly 120) in the form of a pivotable arm 822 is shown. As shown in FIG. 9A, ingestible device 800 is illustrated in a first state prior to ingestible device 800 reaching a desired location in the GI tract for delivering therapeutic preparation 125 into the GI lumen wall. Ingestible device 800 further includes an enclosure 802 (an embodiment of enclosure 102) and an outer coating 804 (an embodiment of outer coating 104.) Enclosure 802 is structured as a capsule having a first segment 802a detachably coupled to a second segment 802b.

Ingestible device 800 further includes a support 815 (an embodiment of support 115). Support 815 is a generally elongated member that defines a generally longitudinal axis 801 of ingestible device 800. Support 815 includes a support first end 815a and a support second end 815b located opposite support first end 815a. Support 815 may have any one of, or a combination of, the structural configurations of support 115 previously described with reference to FIGS. 3A-7B.

Ingestible device 800 further includes a delivery assembly 820 (an embodiment of delivery assembly 120) pivotably coupled to support 815. In the embodiment shown in FIG. 9A, ingestible device 800 includes two delivery assemblies 820 positioned on opposite sides of support 815, although it should be appreciated that ingestible device 800 may include one or more, two or more, three or more, or four or more delivery assemblies 820 according to other embodiments. Each delivery assembly 820 includes a pivotable arm 822. Pivotable arm 822 is shown a substantially linear member, but pivotable arm 822 may be substantially non-linear or include substantially non-linear portions, according to other embodiments. A first end of pivotable arm 822 is pivotably coupled to support 815 at or near support second 815b via a first pivot joint 824a. An opposite, second end of pivotable arm 822 is pivotably coupled to delivery structure 128 including one or more therapeutic preparations 125 therein by a second pivot joint 824b. Delivery structure 128 and therapeutic preparation 125 are shown in block diagrammatic form in FIGS. 9A-9B, but various embodiments of delivery structure 128 and therapeutic preparation 125 are discussed below with reference to FIGS. 10A-13B.

Still referring to FIG. 9A, ingestible device 800 further includes a wedge 825 movably coupled to support 815. Wedge 825 has a generally frusto-conical shape and is movable (e.g., slidable) relative to a surface of support 815 such that wedge can translate relative to support 815 along longitudinal axis 801. As discussed in greater detail below, ingestible device 800 is structured such that actuation of actuator 810 causes wedge 825 to translate axially along longitudinal axis 801 relative to support 815 toward support second end 815b to thereby cause each delivery assembly 820 to pivot about first pivot axis 824a and extend outwardly away from support 815 toward a GI lumen wall for delivery of therapeutic preparation 125.

For example, referring to FIG. 9B, ingestible device 800 is shown in a second state when ingestible device 800 has reached a desired location in the GI tract of a subject to deliver therapeutic preparation 125 from delivery structure 828 into the GI lumen wall of the subject. As shown in FIG. 9B, enclosure 802 and/or outer coating 804 are partially (or fully) degraded as a result of ingestible device 800 having reached the desired location in the GI tract, which, for example, is associated with a selected or threshold pH value to trigger degradation of enclosure 802 and/or outer coating 804. As a result of at least partial degradation of enclosure 802 and/or outer coating 804, fluid in the GI tract is permitted to reach an interior of enclosure 802 to contact release 108 so as to activate (e.g., degrade) release 108. In response to activation of release 108, actuator 110 is actuated to cause extender 130 to extend axially along longitudinal axis 801 and to cause wedge 825 to translate axially relative to support 815 along longitudinal axis 801 toward support second end 815b (as indicated by unidirectional arrow 836.) Wedge 825 can thereby contact and push pivotable arms 722 to cause pivotable arms 722 to pivot about first pivot joints 724a (as indicated by rotational arrow 838) and extend outwardly away from support 815, similar to the function of an umbrella. The frusto-conical shape of wedge 825 can, advantageously, function to push the pivotable arms 722 outwardly as wedge 825 moves axially toward support second end 815b. The outward movement of pivotable arms 822 causes delivery structures 828 to be positioned adjacent (or in contact with) the GI lumen wall to permit delivery of therapeutic preparation 125 into the lumen wall (see, e.g., FIGS. 10A-13B and related discussion.) Further, the outward movement of pivotable arms 822 can assist with the separation of first and second segments 802a, 802b of capsule 802 (as indicated by bidirectional arrow 839.)

Each of the delivery assemblies 720, 820 may include a retaining feature (e.g., a hook, a barb, a sharp protrusion) coupled to, or integrally formed with, each delivery structure 128 for engaging with the GI lumen wall to reorient delivery structures 128 relative to the lumen wall to facilitate delivery of therapeutic preparation 125. For example, the retaining feature of each delivery structure 128 may engage with the tissue of the lumen wall to temporarily hold delivery structures 128 relative to the lumen wall. Temporary retention of delivery structures 128 can cause delivery structures 128 to pivot relative to pivotable arms 722, 822 via second pivot joints 724b, 824b, respectively, to thereby reorient the delivery structure 128 relative to the lumen wall as ingestible device 700, 800 moves through the GI tract (e.g., via peristaltic action of the GI lumen.) Delivery structures 128 may reorient in such a manner that therapeutic preparation 125 can be delivered in a substantially orthogonal direction into the GI lumen wall upon ejection from delivery structures 128. Further, temporary retention of delivery structures 128 may help to provide for sufficient time for ejecting therapeutic preparation 125 into the lumen wall.

Referring generally to FIGS. 10A-13B, various embodiments of delivery structure 128 are shown. It should be appreciated that the various delivery structures described in the following paragraphs can be used in conjunction with any one of, or a combination of, the embodiments of delivery assembly 120 previously discussed herein (e.g., delivery assembly 220, 320, 420, 520, 620, 720, 820.) For example, the various delivery structures discussed herein may be coupled to, or integrally formed with, a platform (e.g., platform 226, 326, 426, 526, 626). In other embodiments, the various delivery structures may be pivotably coupled directly to a pivotable arm (e.g., pivotable arm 722, 822). Further, while the following delivery structures are depicted with a single therapeutic preparation disposed therein, the various delivery structures may be structured to contain a plurality of therapeutic preparations for delivery into a GI lumen wall or surrounding tissue thereof.

In the following embodiments, therapeutic preparation 125 is structured as an elongated member with a tapered end for piercing a seal of a container and penetrating into the GI lumen tissue. A second end opposite the tapered end of therapeutic preparation 125 defines an engagement surface for engaging with an actuating mechanism/second actuator (e.g., protrusion, piston) of delivery structure 128 to eject therapeutic preparation 125 from the container. The engagement surface may be generally planar or may include a structure that is complementary in shape to a portion of the actuating mechanism so as to facilitate a transfer of force from the actuating mechanism to therapeutic preparation 125. It should be appreciated that therapeutic preparation 125 may be structured differently according to other embodiments and is not limited to the following description.

FIG. 10A illustrates a side cross-sectional view of delivery structure 140 (an embodiment of delivery structure 128) for use in one or more embodiments of delivery assembly 120 in the present disclosure. Delivery structure 140 includes a holder 142, a container 144, and a therapeutic preparation 145 (an embodiment of therapeutic preparation 125) disposed within container 144. Delivery structure 140 is shown in a first state prior to reaching a desired location in the GI tract to deliver therapeutic preparation 145 into a GI lumen wall. As discussed below, delivery structure 140 is structured to deliver therapeutic preparation 145 from container 144 into the GI lumen wall. For example, delivery structure 140 relies on an external force applied to delivery structure 140 to eject therapeutic preparation 145 into the lumen wall instead of using a separate actuator for actively delivering therapeutic preparation 145.

Still referring to FIG. 10A, holder 142 includes a perimeter wall 142a defining an interior 142c. Holder 142 further includes a protrusion 142b extending from a lower interior portion of perimeter wall 142a within interior 142c. Protrusion 142b is sized and positioned within interior 142c so as to define a circumferential opening 142c′ surrounding the sides of protrusion 142b. Holder 142 further includes an open end 142d opposite the protrusion 142b for receiving container 144 therethrough and into interior 142c. Protrusion 142b includes a tapered end (e.g., pointed) for piercing a seal of container 142 to eject therapeutic preparation 145 therefrom into a GI lumen wall, as discussed in greater detail below. Protrusion 142b extends to a height below open end 142d such that protrusion 142b is disposed within interior 142c. Holder 142 further includes one or more nubs 142e, or other structural feature, extending inwardly from an inner side portion of perimeter wall 142a. Nubs 142e are structured to provide an interference fit with container 144 to temporarily hold container 144 in place relative to holder 142.

Container 144 is structured to contain therapeutic preparation 145 therein and to temporarily protect therapeutic preparation 145 from degradation within the GI tract. Container 144 is further structured to be movably coupled to holder 142. Container 144 includes a body 144a defining an interior 144a′, a first open end 144a″, and a second open end 144a′″ located opposite first open end 144a″. Therapeutic preparation 145 is disposed within interior 144a′. A first seal 144b is coupled to body 144a at first open end 144a″ and a second seal 144c is coupled to body 144a at second open end 144a′″ such that interior 144a′ is substantially sealed from fluid to temporarily protect therapeutic preparation 145 from degradation. First seal 144b and second seal 144c are formed from a pierceable material to allow for protrusion 142b and therapeutic preparation 145 to puncture/pierce therethrough. For example, first and second seals 144b, 144c can be a pierceable film, such as aluminum foil, which is coupled (e.g., using adhesive) to body 144a.

Body 144a is movably (e.g., slidably) coupled to an inner side portion of perimeter wall 142a. Container 144 is positioned at least partially in interior 142c such that second seal 144c is positioned adjacent the tapered end of protrusion 142b. Container 144 extends outwardly from holder 142 through opening 142d such that first seal 144b defines an outermost portion of delivery structure 140 in the first state shown in FIG. 10A. Nubs 142e can engage with complementary features (e.g., a recess, an opening, a protrusion) on container 144 to temporarily hold container 144 in place relative to holder 142 via an interference condition. As discussed below, container 144 is structured to move (e.g., slide) relative to holder 142 in response to contact with the GI lumen wall to deliver therapeutic preparation 145 from container 144 into the GI lumen wall.

For example, referring to FIG. 10B, delivery structure 140 is shown in a second state when delivery structure 140 is moved into contact with the GI lumen wall via outward extension of delivery assembly 120 (upon actuation of actuator 110, as indicated by unidirectional arrow 146.) The return force from container 144 contacting the GI lumen wall is sufficient to overcome the interference condition between container 144 and nubs 142e of holder 142, such that container 144 is moved inward within interior 142c toward protrusion 142b. This inward movement of container 144 causes protrusion 142b to pierce through second seal 144c and to contact the engagement surface of therapeutic preparation 145 to thereby eject therapeutic preparation 145 from container 144 through first seal 144b into the GI lumen wall or surrounding tissue thereof, where one or more therapeutic agents of therapeutic preparation 145 can release into the subject's bloodstream. Container 144 is structured to fit at least partially within circumferential opening 142c′ to allow for sufficient travel of protrusion 142b within container 144 to eject therapeutic preparation 145 from container 144. In this manner, delivery structure 140 can deliver therapeutic preparation 145 into a GI lumen wall or surrounding tissue thereof.

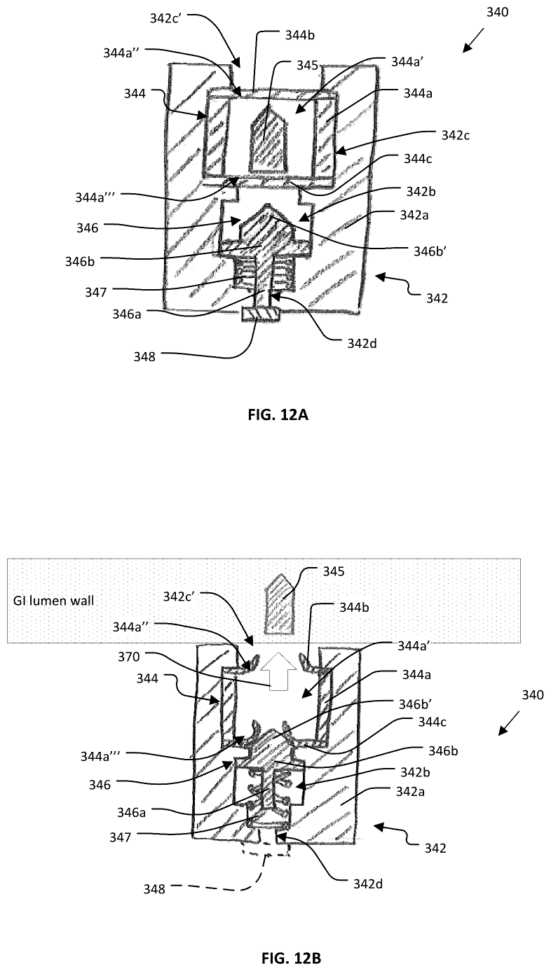

FIG. 11A illustrates a side cross-sectional view of a delivery structure 240 (an embodiment of delivery structure 128) for use in one or more embodiments of delivery assembly 120 in the present disclosure. Delivery structure 240 includes a holder 242, a container 244, a therapeutic preparation 245 (an embodiment of therapeutic preparation 125) disposed within container 244, and an actuating mechanism (e.g., second actuator) including a piston 246. The actuating mechanism further includes an igniter 247, a charge 248, and an electrical circuit 260 (shown in block diagrammatic form) for generating a force to move piston 246. Delivery structure 240 is shown in a first state prior to reaching a desired location in the GI tract to deliver therapeutic preparation 245 into a GI lumen wall. As discussed below, delivery structure 240 is structured to actively deliver, by the actuating mechanism, therapeutic preparation 245 from container 244 into the GI lumen wall. In one or more embodiments, the actuating mechanism of delivery structure 240 may be structured similarly as actuator 410 discussed above with reference to FIGS. 5A-5B.

For example, holder 242 includes a perimeter wall 242a defining an open end 242d′ and an interior including a reaction chamber 242b, a piston chamber 242c, and a container portion 242d. Reaction chamber 242b is defined by a lower inner surface of perimeter wall 242a. Piston chamber 242c is located above reaction chamber 242b and is structured to slidably receive piston 246. Container portion 242d is located above piston chamber 242c adjacent to open end 242d′. Reaction chamber 242b includes igniter 247 and charge 248 disposed therein. Charge 248 is positioned adjacent to and/or in contact with igniter 247. Charge 248 may be a combustible material, such as a composition comprising nitrocellulose (e.g., a nitrocellulose sheet) or other combustible material or combinations of materials. Igniter 247 may be a coil (as shown), a conductive wire or filament (e.g., tungsten filament), or other structure for generating heat, a spark, a flame, or other energy sufficient to ignite charge 248 in response to receiving an electrical current. Holder 242 further includes electrical contacts 249 coupled to perimeter wall 242a. Electrical contacts 249 are operably coupled to igniter 247 and to electrical circuit 260. A release mechanism 249′ (e.g., second release) in the form of a coating or cover is applied to each of the electrical contacts 249 at an outer portion of holder 242 to temporarily prevent electrical contacts 249 from coming into contact with fluids in the GI tract in the first state shown in FIG. 11A. As discussed below, electrical circuit 260 is structured to generate a signal (e.g., a current) in response to an electrical short created between contacts 249 based on contact with bodily fluid in the GI tract when release mechanism 249′ is activated (e.g., degraded.) Electrical circuit 260 is further structured such that igniter 247 initiates combustion of charge 248 in response to the generated signal.

Electrical circuit 260 may be structured similarly as electrical circuit 419 discussed above with reference to FIGS. 5A-5B. For example, electrical circuit 260 may include a power source (e.g., a battery) and one or more energy storage components (e.g., a capacitor) coupled to holder 242. Electrical circuit 260 may further include one or more additional electrical components. Electrical circuit 260 may be structured such that the electrical short across contacts 249 allows for an electrical current from the power source to be stored by the energy storage component. When a sufficient amount of current is accumulated in the energy storage component, the energy storage component can discharge the stored energy through the electrical circuit 260 across igniter 247 thereby causing igniter 247 to initiate combustion of charge 248.

Alternatively, electrical circuit 260 may include one or more sensors for sensing a condition in the GI tract and a processing circuit including a processor for executing instructions stored in a memory of the processing circuit in response to the sensed condition to cause igniter 247 to initiate combustion of charge 248. For example, the one or more sensors may be coupled to holder 242 and may be structured to sense a condition in the GI tract (e.g., a pH value, a temperature, an electrolytic value) to generate a signal. The processor can receive the signal and discharge the current stored in the energy storage component based on information stored in the memory of the processing circuit to thereby cause activation of igniter 247. For example, information may include a threshold pH value, a range of pH values, a threshold temperature, a range of temperatures, or other information relating to a condition in the GI tract.

The electrical circuit 260 may be structured in such a way to cause activation of igniter 247 at an appropriate time relative to actuation of delivery assembly 120 by actuator 110, such that therapeutic preparation 245 can be substantially preserved (e.g., from degradation) for delivery into the GI lumen wall to achieve a desired therapeutic effect in a subject. For example, electrical circuit 260 may include a timer to delay sending a signal to igniter 247 for a sufficient amount of time to enable delivery assembly 120 to extend outwardly to the GI lumen wall before therapeutic preparation 245 is ejected from container 244 into the lumen wall. Alternatively, electrical circuit 260 may include a capacitor that is structured to discharge a current to igniter 247 after accumulating a certain amount of charge which may be associated with the timing of actuation of delivery assembly 120.

In embodiments including an electrical circuit associated with actuator 110 of the device (e.g., electrical circuits 419, 519), electrical circuit 260 may be omitted and the actuating mechanism (e.g., igniter 247) can be operably coupled to the electrical circuit associated with actuator 110 instead. In these embodiments, the electrical circuit associated with actuator 110 may be structured to send a signal to igniter 247 at an appropriate time relative to actuation of delivery assembly 120 to ensure delivery of therapeutic preparation 245 into the GI lumen wall after delivery structure 240 has been positioned in contact with, or adjacent, the GI lumen wall.

Still referring to FIG. 11A, piston 246 is slidably disposed within piston chamber 242c adjacent to reaction chamber 242b. Piston 246 may be slidably coupled to, and sealingly engaged with (e.g., via one or more piston rings, seals), an inner surface of perimeter wall 242a that defines piston chamber 242c. Piston 246 includes a shaft 246a engaged with the inner surface of piston chamber 242c and an end 246b extending from shaft 246a toward container portion 242d. End 246b has a tapered structure (e.g., pointed) that is sufficient for piercing through a seal of container 244 coupled within container portion 242d. End 246b is further structured for contacting an engagement surface of therapeutic preparation 245 to eject therapeutic preparation 245 from container 244 into the GI lumen wall. As discussed below, piston 246 moves axially in piston chamber 242c toward container portion 242d in response to ignition of charge 248 within reaction chamber 242b to deliver therapeutic preparation 245 into the GI lumen wall.

Container 244 is coupled to holder 242 at a fixed position within container portion 242d. For example, holder 242 can include one or more snap features, bayonet features, or other features for retaining container 244 within container portion 242d. Container 244 is structured identically as container 144 discussed above but is fixed relative to holder 242 instead of being movable. As such, like reference numerals refer to like features between embodiments (e.g., body 144a is equivalent to body 244a.) Container 244 contains therapeutic preparation 245 therein and is structured to temporarily protect therapeutic preparation 245 from degradation within the GI tract. Container 244 is positioned relative to holder 242 such that second seal 244c is located adjacent to end 246b of piston 246, and first seal 244b is located adjacent to opening 242d′.

Referring now to FIG. 11B, delivery structure 240 is shown in a second state when delivery structure 240 is moved adjacent to, or in contact with, the GI lumen wall via outward extension of delivery assembly 120 (upon actuation of actuator 110.) In the second state shown in FIG. 11B, fluid in the GI tract is permitted to reach release mechanism 249′ so as to activate (e.g., degrade) release mechanism 249′. In response to activation of release mechanism 249′, bodily fluid in the GI tract creates an electrical short across electrical contacts 249 so as to allow an electrical current to flow through electrical circuit 260. The current generated in electrical circuit 260 is sufficient to cause igniter 247 to ignite charge 248 to generate a combustion force within reaction chamber 242b at an appropriate time relative to actuation of delivery assembly 120 to ensure substantial preservation of therapeutic preparation 245 for delivery into the GI lumen wall. For example, the timing of delivery of therapeutic preparation 245 relative to actuation of delivery assembly 120 may be controlled by a number of factors, such as by the structure of release mechanism 249′ (e.g., degradation rate), structure of electrical circuit 260 (as discussed above), structure of igniter 247, structure/amount of charge 248, and/or structure of piston 246 (e.g., amount of travel, resistance within piston chamber). Additionally or alternatively, activation of release mechanism 249′ may be controlled by temporarily blocking release mechanism 249′ by a component of delivery assembly 120 (e.g., by one or more pivotable arms) such that movement/extension of delivery assembly 120 exposes release mechanism 249′ to allow for activation of release mechanism 249′.

Still referring to FIG. 11B, the force generated in reaction chamber 242b causes piston 246 to move in piston chamber 242c toward first seal 244b. This movement of piston 246 is sufficient to cause end 246b to pierce first seal 244b and to contact an engagement surface of therapeutic preparation 245 to thereby eject therapeutic preparation 245 from container 244 through second seal 244c (as indicated by unidirectional arrow 270) into the GI lumen wall or surrounding tissue thereof, where one or more therapeutic agents of therapeutic preparation 245 can subsequently release into the subject's bloodstream. In this manner, delivery structure 240 can actively deliver therapeutic preparation 245 into a GI lumen wall or surrounding tissue thereof.

FIG. 12A illustrates a side cross-sectional view of a delivery structure 340 (an embodiment of delivery structure 128) for use in one or more embodiments of delivery assembly 120 in the present disclosure. Delivery structure 340 includes a holder 342, a container 344, a therapeutic preparation 345 (an embodiment of therapeutic preparation 125) disposed within container 344, and an actuating mechanism (e.g., second actuator) including a piston 346 and a biasing member in the form of a spring 347. Delivery structure 340 is shown in a first state prior to reaching a desired location in the GI tract to deliver therapeutic preparation 345 into a GI lumen wall. As discussed below, delivery structure 340 is structured to actively deliver, by actuation of spring 347, therapeutic preparation 345 from container 344 into the GI lumen wall.

Holder 342 includes a perimeter wall 342a defining an open end 342c′ and an interior including a piston chamber 342b and a container portion 342c. Piston chamber 342b extends from a lower inner surface of perimeter wall 342a to container portion 342c. Piston chamber 342b is structured to slidably receive piston 346 therein. Container portion 342c is located above piston chamber 342b adjacent to open end 342c′. Spring 347 and piston 346 are arranged in piston chamber 342b such that spring 347 is held in a compressed state between a lower inner surface of perimeter wall 342a and a portion of piston 346 in the first state shown in FIG. 12A. Piston 346 may be slidably coupled to, and sealingly engaged with (e.g., via one or more piston rings, seals), an inner surface of perimeter wall 342a that defines piston chamber 342b.

For example, piston 346 includes a shaft 346a and a piston portion 346b extending from shaft 346a. Piston portion 346b is movably engaged with the inner surface of piston chamber 342b. Piston portion 346b further includes an end 346b′ that is tapered (e.g., pointed) for piercing through a seal of container 344 and for engaging with an engagement surface of therapeutic preparation 345. Shaft 346a is shown disposed through spring 347 and into an opening 342d in perimeter wall 342a such that spring 347 contacts a lower surface of piston portion 346b. A release mechanism 348 (e.g., second release) is coupled to shaft 346a at opening 342d to temporarily hold piston 346 in position relative to holder 342 to thereby compress spring 347 in the first state shown in FIG. 12A. Release mechanism 348 may comprise a degradable material for degrading in response to contact with fluid in the GI tract to cause release of piston 346. Additionally or alternatively, release mechanism 348 may be structured as a latch, a clip, or other structure that can release piston 346 in response to a condition in the GI tract (e.g., a temperature, a pH value.) Additionally or alternatively, release mechanism 348 may be structured to move or open in response to movement of a component of device 100 (e.g., pivotable movement of one or more arms.) As discussed below, spring 347 is structured to expand in response to activation of release mechanism 348 to cause piston 346 to move toward container 344 to deliver therapeutic preparation 345 into a GI lumen wall.

Container 344 is coupled to holder 342 at a fixed position within container portion 342c. For example, holder 342 can include one or more snap features, bayonet features, or other features for retaining container 344 within container portion 342c. Container 344 is structured identically as container 244 discussed above. As such, like reference numerals refer to like features between embodiments (e.g., body 244a is equivalent to body 344a.) Container 344 contains therapeutic preparation 345 therein and is structured to temporarily protect therapeutic preparation 345 from degradation within the GI tract. Container 344 is positioned relative to holder 342 such that second seal 344c is located adjacent to end 346b of piston 346, and first seal 344b is located adjacent to opening 342c′.

Referring now to FIG. 12B, delivery structure 340 is shown in a second state when delivery structure 340 is moved adjacent to, or in contact with, the GI lumen wall via outward extension of delivery assembly 120 (upon actuation of actuator 110.) In the second state shown in FIG. 12B, fluid in the GI tract is permitted to reach release mechanism 348 so as to activate (e.g., degrade and/or release) release mechanism 348. In response to activation of release mechanism 348, the biasing force of spring 347 overcomes the retention force of piston 346 by release mechanism 348 to allow spring 347 to expand and move piston 346 in piston chamber 342b toward container 344. This movement of piston 346 is sufficient to cause end 346b′ to pierce first seal 344b and to contact the engagement surface of therapeutic preparation 345 to thereby eject therapeutic preparation 345 from container 344 through second seal 344c (as indicated by unidirectional arrow 370) into the GI lumen wall or surrounding tissue thereof, where one or more therapeutic agents of therapeutic preparation 345 can release into the subject's bloodstream.

The actuation of spring 347 can be appropriately timed relative to actuation of delivery assembly 120 to ensure substantial preservation of therapeutic preparation 345 for delivery into the GI lumen wall. For example, the timing of delivery of therapeutic preparation 345 relative to actuation of delivery assembly 120 may be controlled by a number of factors, such as by the structure of release mechanism 348 (e.g., degradation rate), structure of spring 347 (e.g., spring constant, size of spring, length of spring), and structure of piston 346 or piston chamber 342b (e.g., amount of piston travel, resistance within piston chamber). Additionally or alternatively, activation of release mechanism 248 may be controlled by temporarily blocking release mechanism 248 by a component of device 100 (e.g., by one or more pivotable arms) such that movement/extension of delivery assembly 120 exposes and/or moves release mechanism 248 to allow for activation of release mechanism 248.

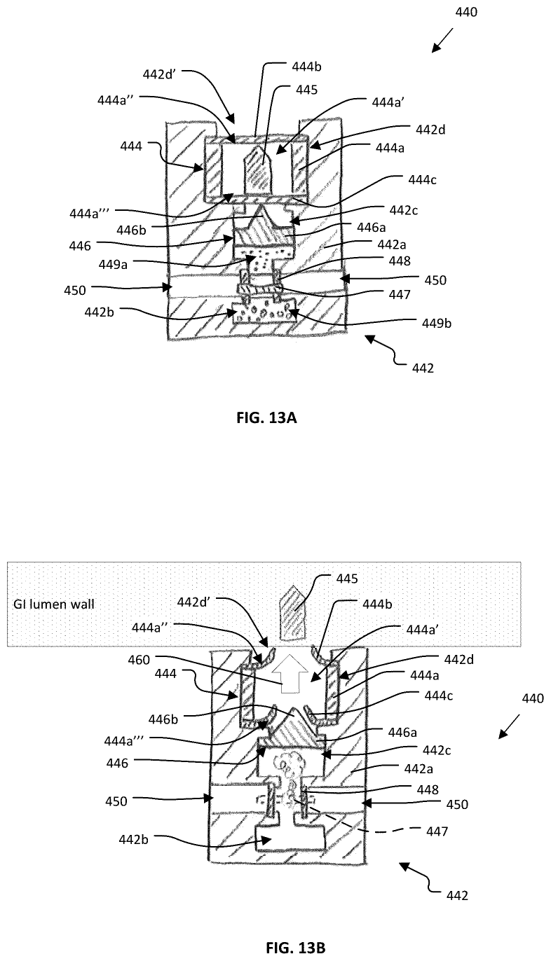

FIG. 13A illustrates a side cross-sectional view of a delivery structure 440 (an embodiment of delivery structure 128) for use in one or more embodiments of delivery assembly 120 in the present disclosure. Delivery structure 440 includes a holder 442, a container 444, a therapeutic preparation 445 (an embodiment of therapeutic preparation 125) disposed within container 444, and an actuating mechanism (e.g., second actuator) including a piston 446. The actuating mechanism further includes a first reactant 449a and a second reactant 449b disposed in respective chambers of holder 442 to be mixed together to generate a gas for moving piston 446. Delivery structure 440 is shown in a first state prior to reaching a desired location in the GI tract to deliver therapeutic preparation 445 into a GI lumen wall. As discussed below, delivery structure 440 is structured to actively deliver, by the actuating mechanism, therapeutic preparation 445 from container 444 into the GI lumen wall.

Holder 442 includes a perimeter wall 442a defining an open end 442d′ and an interior including a reaction chamber 442b, a piston chamber 442c, and a container portion 442d. Reaction chamber 442b is defined by a lower inner surface of perimeter wall 442a and includes a first reactant 449a (e.g., potassium bicarbonate) disposed therein. Piston chamber 442c is located above reaction chamber 442b and is structured to slidably receive piston 446. Piston 446 includes a piston portion 446a that is slidably engaged with an inner surface of perimeter wall 442a that defines piston chamber 442c. Piston 446 further includes an end portion 446a that is tapered (e.g., pointed) for piercing through a seal of container 444 and for engaging with an engagement surface of therapeutic preparation 445.

Piston chamber 442c further includes a second reactant 449b (e.g., citric acid) disposed below piston 446 adjacent reaction chamber 442b. Container portion 442d is located above piston chamber 442c adjacent open end 442d′. Reaction chamber 442b is fluidly coupled to piston chamber 442c by a conduit 448. However, in the first state shown in FIG. 12A, conduit 448 includes a release mechanism 447 (e.g., second release) coupled thereto to temporarily block fluid flow between reaction chamber 442b and piston chamber 442c. For example, conduit 448 may be a flexible tube (e.g., silicone tube) and release mechanism 447 may be a clip, a latch, or other structure that functions as a pinch valve to temporarily block fluid flow through conduit 448. Release mechanism 447 may comprise a degradable material to degrade in response to contact with fluid in the GI tract. Additionally or alternatively, release mechanism 447 may be structured as a latch, a clip, or other structure that can move or otherwise release from conduit 448. Alternatively, release mechanism 447 may be structured as another type of valve member that is movable relative to holder 442, so as to selectively fluidly couple reaction chamber 442b with piston chamber 442c in response to a condition in the GI tract and/or movement of a component of device 100 (e.g., pivotable movement of one or more arms.) Holder 442 further includes one or more openings 450 to provide a fluid pathway from the GI lumen environment to release mechanism 447. As discussed below, release mechanism 447 is structured to release conduit 448 to allow first reactant 449a to flow from reaction chamber 442b to piston chamber 442c where first reactant 449a can mix with second reactant 449b to generate a gas (e.g., CO2) having a pressure sufficient to move piston 446 toward container 444.

Container 444 is coupled to holder 442 at a fixed position within container portion 442d. For example, holder 442 can include one or more snap features, bayonet features, or other features for retaining container 444 within container portion 442d. Container 444 is structured identically as container 244 discussed above. As such, like reference numerals refer to like features between embodiments (e.g., body 244a is equivalent to body 444a.) Container 444 contains one or more therapeutic preparations 445 therein and is structured to temporarily protect therapeutic preparation 445 from degradation within the GI tract. Container 444 is positioned relative to holder 442 such that second seal 444c is located adjacent end 446b of piston 446, and first seal 444b is located adjacent opening 442d′.

Referring to FIG. 13B, delivery structure 440 is shown in a second state when delivery structure 440 is moved adjacent to, or in contact with, the GI lumen wall via outward extension of delivery assembly 120 (upon actuation of actuator 110.) In the second state shown in FIG. 13B, fluid in the GI tract is permitted to reach release mechanism 447 so as to activate (e.g., degrade and/or release) release mechanism 447. In response to activation of release mechanism 447, conduit 448 is at least partially, or fully, opened to allow first reactant 449a to flow from reaction chamber 442b into piston chamber 442c to mix with second reactant 449b to generate a gas (e.g., CO2.) The generated gas has sufficient pressure to cause piston 446 to move in piston chamber 445 toward container 444. This movement of piston 446 is sufficient to cause end 446b to pierce first seal 444b and to contact the engagement surface of therapeutic preparation 445 to thereby eject therapeutic preparation 445 from container 444 through second seal 444c (as indicated by unidirectional arrow 460) into the GI lumen wall or surrounding tissue thereof, where one or more therapeutic agents of therapeutic preparation 445 can subsequently release into the subject's bloodstream.

The delivery of therapeutic preparation 445 from container 444 can be appropriately timed relative to actuation of delivery assembly 120 to ensure substantial preservation of therapeutic preparation 445 for delivery into the GI lumen wall. For example, the timing of delivery of therapeutic preparation 445 relative to actuation of delivery assembly 120 may be controlled by a number of factors, such as by the structure of release mechanism 447 (e.g., degradation rate), the structure of conduit 448 (e.g., length, internal diameter), amount or type of reactants 449a, 449b, and structure of piston 446 or piston chamber 442b (e.g., amount of piston travel, resistance within piston chamber.) Additionally or alternatively, activation of release mechanism 447 may be controlled by temporarily blocking release mechanism 447 by a component of device 100 (e.g., by one or more pivotable arms) such that movement/extension of delivery assembly 120 exposes and/or moves release mechanism 447 to allow for activation of release mechanism 447.

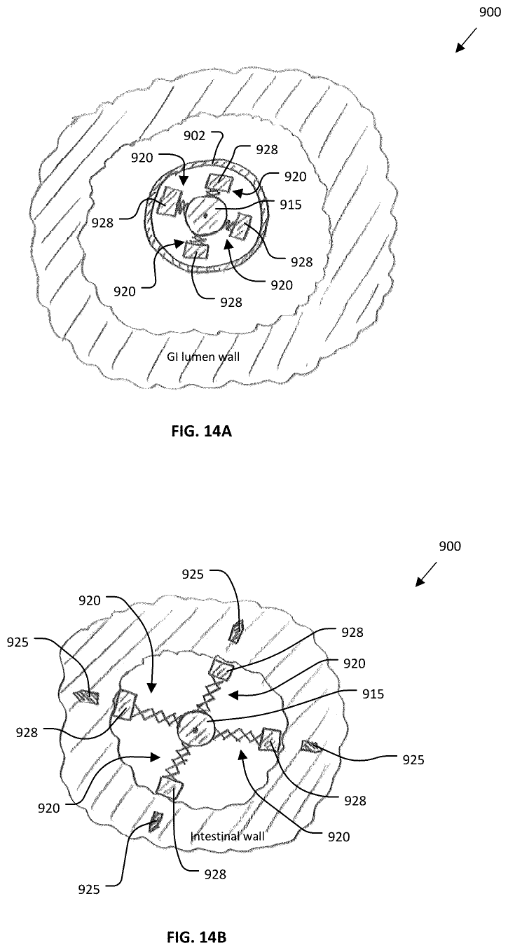

FIG. 14A illustrates a front cross-sectional view of an ingestible 900 (an embodiment of ingestible device 100) located within a lumen of a GI tract of a subject after being ingested. Ingestible device 900 is shown in a first state prior to reaching a desired location in the GI tract for delivery of a therapeutic preparation into the GI lumen wall. As shown in the front view of FIG. 14A, ingestible device 900 includes an enclosure 902 (an embodiment of enclosure 102), a support 915 (an embodiment of support 115) disposed in the enclosure 902, and a plurality of delivery assemblies 920 (an embodiment of delivery assembly 120) coupled to, and positioned circumferentially around, support 915. Support 915 is shown to have a circular cross-sectional shape, but it should be appreciated that support 915 may have any suitable cross-sectional shape (e.g., ellipsoid, triangular, square, pentagonal, hexagonal.) Ingestible device 900 is shown to include four delivery assemblies 920, but ingestible device 900 may include more or fewer than four delivery assemblies 920 according to other embodiments. Delivery assemblies 920 are positioned equidistant relative to each other but may be spaced differently according to other embodiments. Each of the delivery assemblies 920 includes a delivery structure 928 (an embodiment of delivery structure 128) containing one or more therapeutic preparations 925 (an embodiment of therapeutic preparation 125) therein for delivery into a GI lumen wall.

Referring to FIG. 14B, ingestible device 900 is shown in a second state when ingestible device 900 has reached a desired location in the GI tract, which in this example is the small intestine, for delivery of therapeutic preparations 925 from delivery structures 928 into the intestinal wall. As shown in FIG. 14B, enclosure 902 is at least partially (or fully) degraded as a result of ingestible device 900 having reached the small intestine, which, for example, is associated with a selected or threshold pH value associated with the small intestine to trigger degradation of enclosure 902. As a result of at least partial degradation of enclosure 902, fluid in the small intestine is permitted to reach an interior of enclosure 902 to contact a release (not shown) associated with an actuator (not shown) of the device so as to activate (e.g., degrade) the release. In response to activation of the release, the actuator causes delivery assemblies 920 to extend outwardly away from support 915 toward the intestinal wall such that delivery structures 928 can deliver therapeutic preparations 925 into the intestinal wall or surrounding tissue thereof (e.g., peritoneum or peritoneal cavity), where one or more therapeutic agents of therapeutic preparations 925 can subsequently release into the subject's bloodstream.

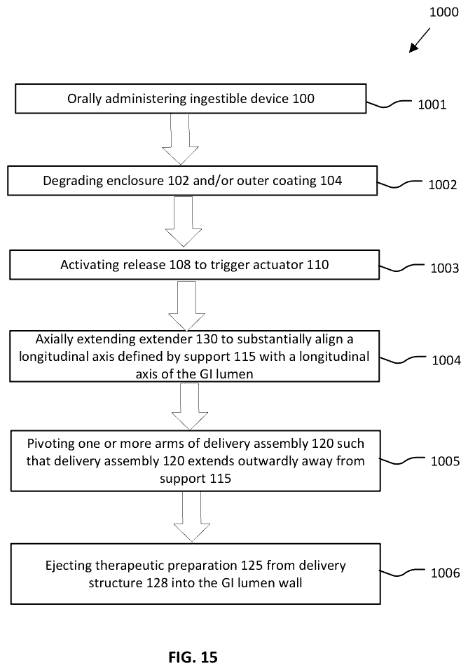

Referring to FIG. 15, a method 1000 of delivering therapeutic preparation 125 into the GI lumen wall or surrounding tissue of a subject using ingestible device 100 is illustrated in diagrammatic form. In a first step 1001, ingestible device 100 is orally administered to the subject by swallowing the ingestible device 100. In a second step 1002, enclosure 102 and/or outer coating 104 are at least partially (or fully) degraded as a result of ingestible device 100 having reached a desired location in the GI tract (e.g., stomach, small intestine) of the subject, which, for example, is associated with a selected or threshold pH value to trigger degradation of enclosure 102. In a third step 1003, fluid in the GI tract is permitted to reach an interior of enclosure 102 to contact release 108 so as to activate release 108 and trigger actuator 110. In a fourth step 1004, actuator 110 causes extender 130 to extend axially along a longitudinal axis defined by support 115 to substantially align the longitudinal axis with a longitudinal axis of the GI lumen. In a fifth step 1005, actuator 110 further causes one or more arms to pivot such that delivery assembly 120 extends outwardly away from support 115 toward the GI lumen wall. In one or more embodiments, step 1005 is performed substantially simultaneously with step 1004. In other embodiments, step 1005 is performed after step 1004. In a sixth step 1006, delivery structure 128 ejects (e.g., using delivery structure 140) therapeutic preparation 125 without a separate actuating mechanism into the GI lumen wall or surrounding tissue thereof in response to delivery structure 128 contacting the GI lumen wall when delivery assembly 120 is extended outwardly. Additionally or alternatively, delivery structure 128 actively ejects (e.g., using delivery structure(s) 240, 340, 440) therapeutic preparation 125 into the GI lumen wall or surrounding tissue thereof in response to a separate release and actuating mechanism associated with delivery structure 128. Therapeutic preparation 125 can subsequently degrade in the lumen wall or surrounding tissue thereof to release one or more therapeutic agents into the subject's bloodstream so as to provide a desired therapeutic effect.

The foregoing description of various embodiments has been presented for purposes of illustration and description. It is not intended to limit the invention to the precise forms disclosed. Many modifications, variations and refinements will be apparent to practitioners skilled in the art. For example, embodiments of the device can be sized and otherwise adapted for various pediatric and neonatal applications as well as various veterinary applications. Also, those skilled in the art will recognize, or be able to ascertain using no more than routine experimentation, numerous equivalents to the specific devices and methods described herein. Such equivalents are considered to be within the scope of the present disclosure.

While the present disclosure has been described and illustrated with reference to specific embodiments thereof, these descriptions and illustrations do not limit the present disclosure. It can be clearly understood that various changes can be made, and equivalent components can be substituted within the embodiments, without departing from the true spirit and scope of the present disclosure as defined by the appended claims. Also, components, characteristics, or acts from one embodiment can be readily recombined or substituted with one or more components, characteristics or acts from other embodiments to form numerous additional embodiments within the scope of the invention. Moreover, components that are shown or described as being combined with other components, can, in various embodiments, exist as standalone components. Further, for any positive recitation of a component, characteristic, constituent, feature, step or the like, embodiments of the invention specifically contemplate the exclusion of that component, value, characteristic, constituent, feature, step or the like. The illustrations may not necessarily be drawn to scale. There can be distinctions between the artistic renditions in the present disclosure and the actual apparatus, due to variables in manufacturing processes and such. There can be other embodiments of the present disclosure which are not specifically illustrated. The specification and drawings are to be regarded as illustrative rather than restrictive. Modifications can be made to adapt a particular situation, material, composition of matter, method, or process to the objective, spirit and scope of the present disclosure. All such modifications are intended to be within the scope of the claims appended hereto. While the methods disclosed herein have been described with reference to particular operations performed in a particular order, it can be understood that these operations can be combined, sub-divided, or re-ordered to form an equivalent method without departing from the teachings of the present disclosure. Therefore, unless specifically indicated herein, the order and grouping of the operations are not limitations of the present disclosure.

1. An ingestible device for delivering a therapeutic preparation into a lumen wall or a surrounding tissue of a gastrointestinal (GI) tract of a subject, the device comprising:
an enclosure;
a support disposed in the enclosure, the support defining a longitudinal axis;
an arm pivotably coupled to the support;
a delivery assembly coupled to the support, the delivery assembly comprising:
a delivery structure coupled to the arm; and
a therapeutic preparation disposed in the delivery structure;
an extender coupled to at least one of the support or the delivery assembly, the extender structured to extend along the longitudinal axis so as to substantially align the delivery structure relative to the lumen wall;
an actuator coupled to the delivery assembly; and
a release coupled to the actuator, the release structured to activate in response to a condition in the GI tract so as to actuate the actuator;
wherein, in response to activation of the release, the actuator causes the arm to pivot relative to the support such that the delivery assembly extends outwardly away from the longitudinal axis toward the lumen wall to deliver the therapeutic preparation from the delivery structure into the lumen wall or the surrounding tissue.

2. The device of claim 1, wherein the actuator is structured to generate an axial force along the longitudinal axis to cause the arm to pivot.

3. The device of claim 1, wherein the actuator is structured to generate a rotational force about the longitudinal axis to cause the arm to pivot.

4. The device of claim 1, wherein the support includes a plurality of sections movably coupled together to define a telescoping arrangement such that when the release is activated, the plurality of sections move relative to each other such that the support extends axially along the longitudinal axis.

5. The device of claim 1, wherein the actuator is coupled to the delivery assembly and to the extender such that when the release is activated, the actuator substantially simultaneously causes the delivery assembly to extend outwardly away from the longitudinal axis and the extender to extend along the longitudinal axis.

6. The device of claim 1, wherein the enclosure defines an interior, and wherein the support, the arm, the delivery assembly, the extender, the actuator, and the release are disposed within the interior.

7. The device of claim 1, wherein the enclosure is structured as a capsule including a first segment and a second segment, wherein the first segment is detachably coupled to the second segment.

8. The device of claim 7, wherein at least one of the actuator, the support, or the extender is structured to push the first and second segments apart.

9. The device of claim 1, wherein the release is a first release and the actuator is a first actuator, and wherein the delivery structure further includes a second release operably coupled to a second actuator for actively delivering the therapeutic preparation.

10. The device of claim 9, wherein the second release is structured to cause the second actuator to eject the therapeutic preparation from the delivery structure into the GI lumen wall or surrounding tissue after the first actuator causes the delivery assembly to extend outwardly away from the support.

11. The device of claim 9, wherein the second release is selectively exposed to fluid in the GI lumen in response to movement of the delivery assembly so as to activate the second release.

12. (canceled)

13. The device of claim 9, wherein the first release has a first degradation rate and the second release has a second degradation rate, and wherein the second degradation rate is less than the first degradation rate.

14. The device of claim 1, wherein the delivery structure includes a holder coupled to the arm and a container coupled to the holder, and wherein the therapeutic preparation is disposed in the container.

15-16. (canceled)

17. The device of claim 14, wherein the holder defines a piston chamber and the delivery structure includes a piston slidably disposed in the piston chamber.

18-19. (canceled)

20. The device of claim 17, wherein the delivery structure further includes a spring coupled to the piston and a release mechanism coupled to the spring, and wherein upon activation of the release mechanism, the spring expands to cause the piston to eject the therapeutic preparation from the container into the lumen wall.

21. The device of claim 1, wherein the delivery structure includes a retaining feature for temporarily retaining the delivery structure relative to the lumen wall.

22. (canceled)

23. The device of claim 1, wherein the actuator includes a spring, and wherein the spring is held in a compressed state by the release such that upon activation of the release, the spring expands along the longitudinal axis to cause the arm to pivot relative to the support.

24. The device of claim 1, wherein the actuator includes an inflatable member structured to function as the extender, wherein the inflatable member includes a plurality of reactants disposed therein, wherein the reactants are temporarily separated from each other by the release, and wherein the release is coupled to the inflatable member such that upon activation of the release, the plurality of reactants mix together to cause a chemical reaction within the inflatable member to create a gas to cause the inflatable member to expand and thereby cause the arm to pivot relative to the support.

25. The device of claim 1, wherein the actuator includes a housing defining a piston chamber, and wherein the support includes a piston slidably disposed within the piston chamber.

26. (canceled)

27. The device of claim 25, wherein the actuator further includes a hydrogel disposed in the piston chamber, wherein the release is coupled to the housing, wherein the housing includes a plurality of openings to provide a fluid path between the piston chamber and the GI lumen environment, and wherein upon activation of the release, fluid from the GI lumen environment enters the piston chamber through one or more of the plurality of openings to cause the hydrogel to expand within the piston chamber to move the piston and the support to thereby cause the arm to pivot relative to the support.

28-39. (canceled)
